(19) United States
(12) Patent Application Publication
(10) Pub. No.: US 2004/0209809 A1
(43) Pub. Date: Oct. 21, 2004

(54) Shc modulation and uses thereof

(76) Inventors: Caroline Saucier, Verdun (CA); Morag Park, Montreal (CA); Anthony J. Pawson, Toronto (CA); Ka-Man Venus Lai, Elmsford, NY (US)

Correspondence Address: Licata & Tyrrell P.C., 66 E. Main Street, Marlton, NJ, 08053, US

(21) Appl. No.: 10/782,283

(22) Filed: Feb. 19, 2004

Related U.S. Application Data
(60) Provisional application No. 60/447,709, filed on Feb. 19, 2003.

(51) Int. Cl. A61K 38/18

(52) U.S. Cl. 514/12

(57) ABSTRACT

The present invention relates to methods for modulating angiogenesis via regulating Shc activity or expression so that VEGF production is altered. Methods for preventing or treating an angiogenesis-related disease or condition as well as methods for identifying an agent for preventing or treating an angiogenesis-related disease or condition are also provided.

### INTRODUCTION

This application claims the benefit of priority from U.S. patent application Ser. No. 60/447,709 filed Feb. 19, 2003, which is incorporated herein by reference in its entirety.

### BACKGROUND OF THE INVENTION

Based on their involvement in a number of key cellular processes, signal transduction pathways and signaling molecules have been the focus of much study. As in many cases perturbation of such processes is associated with disease, such pathways are of particular interest to elucidate mechanisms of disease, and to identify possible targets for therapeutic intervention or diagnostics. In particular, a number of such signaling molecules have been implicated in cancer (see, e.g., Blume-Jensen and Hunter (2001) Nature 411:355-365). Such signaling molecules include cell surface receptor molecules as well as intracellular signaling proteins, which may directly possess binding and/or catalytic activity (e.g. kinase or phosphatase activity), in some cases acting as adaptor- or scaffold-type molecules capable of binding other signaling molecules to provide a link in a pathway.

Examples of the above-noted signaling molecules include receptor tyrosine kinase (RTK) molecules. Among the 58 members of the RTK family which have been identified, oncogenic deregulation of at least 30 RTKs has been linked to various human malignancies. The mechanisms that lead to deregulation of RTKs may differ, but in all cases, the tightly regulated intracellular signaling of the RTK is perturbed (Blume-Jensen and Hunter (2001) supra). Deregulation of a receptor or physiological stimulation by a ligand promotes activation of the intracellular kinase domain and phosphorylation of the receptor on tyrosine residues that act as binding sites for a variety of signaling proteins. These proteins contain Src homology 2 (SH2) or phosphotyrosine binding (PTB) domains that recognize phosphorylated tyrosine residues in the context of their surrounding amino acids (Pawson and Nash (2000) Genes Dev. 14:1027-1047). The combination of proteins recruited to RTKs dictates a series of downstream signals within the interior of the cell that culminate in distinct biological effects.

Shc is an adaptor-type intracellular signaling protein containing both SH2 and PTB domains (Blaikie (1994) J. Biol. Chem. 269:32031-32034; van der Geer (1995) Curr. Biol. 5:404-412; Pelicci (1996) Oncogene 13:633-641).

Shc is capable of binding to phosphorylated tyrosine residues of the cytoplasmic domain of RTK's (by virtue of its PTB or SH2 domains), as well as to other intracellular signaling molecules, thus playing a role in the intracellular transduction of an RTK-derived signal. Several studies have implicated the recruitment of Shc or the adaptor protein Grb2 as important mediators of cell transformation downstream from RTKs (Fixman, et al. (1996) J. Biol. Chem. 271:13116-13122; Ponzetto, et al. (1996) J. Biol. Chem. 271:14119-14123; Dankort, et al. (2001) Mol. Cell. Biol. 21:1540-1551; Asai, et al. (1996) J. Biol. Chem. 271:17644-17649). The Grb2 and Shc adaptor proteins associate with tyrosine phosphorylated RTKs through their respective SH2 and PTB domains (Lowenstein, et al. (1992) Cell 70:431-442; Rozakis-Adcock, et al. (1993) Nature 363:83-85; van der Geer, et al. (1995) Curr. Biol. 5:404-412; Batzer, et al. (1995) Mol. Cell. Biol. 15:4403-4409). Moreover, the recruitment of Shc to activated RTKs results in its phosphorylation on tyrosine residues Tyr^{239/240} and Tyr³¹⁷ (Gotoh, et al. (1996) EMBO J. 15:6197-6204; van der Geer, et al. (1996) Curr. Biol. 6:1435-1444). These tyrosines provide optimal binding sites for the SH2 domain of Grb2 and several RTKs rely on Shc to indirectly recruit Grb2 (van der Geer, et al. (1996) supra). In turn, Grb2 through protein interactions with its SH3 domains links the receptor with multiple downstream signaling proteins. Disruption of Shc in mice is found to be lethal (Lai & Pawson (2000) Genes & Dev. 14:1132-1145).

### SUMMARY OF THE INVENTION

One aspect of the present invention is a method for modulating angiogenesis in a cell, tissue, or subject. The method involves contacting a cell, tissue, or subject with an agent which regulates the expression or activity of Shc thereby altering the production of VEGF or the expression of a modulator of angiogenesis so that angiogenesis in said cell, tissue, or subject is modulated. In particular embodiments, the modulator of angiogenesis is fibroblast growth factor-2, angiopoietin-2, thombospondin-1 or angiopoietin-1.

Another aspect of the present invention is a method for preventing or treating an angiogenesis-related disease or process in a subject. The method involves administering to a subject an agent which modulates the expression or activity of Shc thereby altering the production of VEGF or expression of a modulator of angiogenesis so that the angiogenesis-related disease or process in said subject is prevented or treated. In particular embodiments, the modulator of angiogenesis is fibroblast growth factor-2, angiopoietin-2, thombospondin-1 or angiopoietin-1.

A further aspect of the present invention, is a method for identifying an agent that modulates angiogenesis. The method involves contacting a first cell expressing Shc with a test agent and measuring expression of a modulator of angiogenesis in said first cell as compared to a second cell expressing Shc not contacted with the test agent, wherein a lower or higher measured activity in the first cell, as compared to the measured activity in the second cell is indicative of an agent which modulates angiogenesis. In embodiments of the invention, the modulator of angiogenesis is VEGF, fibroblast growth factor-2, angiopoietin-2, thombospondin-1 or angiopoietin-1.

### BRIEF DESCRIPTION OF THE DRAWINGS

FIG. 1 depicts the RTK oncoproteins specific for the binding of Grb2 or Shc. The amino acid sequences substituted within the Tpr-Met Tyr^{482/489}Phe cassette mutant and the inserted binding motifs are shown. The Grb2 binding site from the EGFR was inserted to generate the Y-Grb2 binding variant, whereas the Y-Shc-1 and Y-Shc-2 variants contain respectively the Shc binding sites from the TrkA or EGF receptors (Saucier, et al. (2002) Oncogene 21:1800-1811).

FIG. 2 depicts RTKs specific for the recruitment of Grb2 or Shc.

FIG. 3 depicts the activated Neu RTK (NT, V664E mutant) that either lacks all known tyrosine autophosphorylation sites (Neu Tyrosine Phosphorylation Deficient, NYPD) or derived add-back mutants containing a single tyrosine phosphorylation binding site for either the adaptor Grb2 (NT-YB, 1144) or Shc (NT-YD, 1226) (Dankort, et al. (1997) Mol. Cell. Biol. 17(9):5410-25).

### DETAILED DESCRIPTION OF THE INVENTION

RTKs modulate a wide range of cellular processes and their deregulation contributes to many hallmarks of cancer, including unrestrained cell proliferation, morphological transformation, anchorage-independent growth, evasion of apoptosis, cell motility, invasion, and angiogenesis (Blume-Jensen & Hunter (2001) Nature 411:355-365). To identify receptor-derived signals that contribute to these processes, RTKs and derived-oncoproteins designed to bind to a single signaling protein were generated. It was shown that the recruitment of Shc, but not the Grb2 adaptor protein, to an activated RTK, promoted VEGF mRNA accumulation and protein production.

The studies described herein exploited the uncharacteristic signaling mechanism of the hepatocyte growth factor RTK (Met)-derived oncoprotein, Tpr-Met. For most RTKs, multiple tyrosine residues located outside of their catalytic domains are required for the recruitment of independent signaling proteins (Pawson & Nash (2000) Genes Dev. 14:1027-1047). In contrast, the biological activity and the recruitment of signaling proteins by the Met receptor, and Tpr-Met oncoprotein, are dependent on twin tyrosines in the carboxy-terminus of the receptor (Met Y1349/1356; Tpr-Met Y482/489)(Fixman, et al. (1996) J. Biol. Chem. 271:13116-13122; Fixman, et al. (1997) J. Biol. Chem. 272:20167-20172; Ponzetto, et al. (1996) J. Biol. Chem. 271:14119-14123; Weidner, et al. (1996) Nature 384:173-176). Using this system, signaling-specific RTK oncoproteins were engineered whereby the twin tyrosines within the Tpr-Met oncoprotein were substituted with a cassette encoding a tyrosine-based motif specific for the binding of a single signaling protein. Using these tools, it has now been shown that the direct recruitment of the adaptor proteins Grb2 or Shc to a RTK oncoprotein is sufficient to induce similar parameters of cell transformation in vitro, including foci of morphologically transformed fibroblasts, anchorage-independent growth, and experimental metastasis, thus indicating that both Grb2 and Shc play similar roles in such processes (Saucier, et al. (2002) Oncogene 21:1800-1811).

Cells within the human body require for their survival oxygen and nutrients. As a consequence, cells within an organism are localized within the mean oxygen diffusion distance (100-200 μm) of a capillary blood vessel. During embryonic development, angiogenesis, the process of new blood vessel growth/formation (neovascularization), occurs to accommodate newly forming and growing organs. This process is controlled by the balance of pro-angiogenic and anti-angiogenic factors that act principally on endothelial cells that are the main components of blood vessels. During the adult life of a healthy subject, stimulation of angiogenesis is limited and takes place when required, such as during wound healing, exercised muscle, and during the menstruation cycle. However, angiogenesis has also been linked to disease, both in cases where it is excessive or insufficient. For example, ocular neovascularization in diseases such as age-related macular degeneration and diabetic retinopathy constitute one of the most common causes of blindness. Intimal hyperplasia causing restenosis or narrowing of the artery has been found to occur in 30-50% of coronary angioplasties and following approximately 20% of bypass procedures (McBride, et al. (1988) N. Engl. J. Med. 318:1734; Clowes (1986) J. Vasc. Surg. 3:381). In cancer, angiogenesis induced by solid tumor growth may lead not only to enlargement of the primary tumor, but also to metastasis via the new vessels. Angiogenesis has also been implicated in rheumatoid arthritis, psoriasis, arteriosclerosis, purogenic granuloma, scleroderma, trachoma, and endometriosis, hemangiomas and other conditions.

Many of these pathological conditions depend on neovascularization for their development. For example, solid tumors, will not expand beyond a size of ˜2 mm³ if new blood vessels from the preexisting host vasculature are not attracted to supply oxygen and nutrients required to sustain their growth (Folkman (1971) N. Engl. J. Med. 285:1182-1186; Carmeliet & Jain (2000) Nature 407:249-257; Folkman (1995) N. Engl. J. Med. 333:1757-1763). In endometriosis, the growth of endometrial lesions outside of the uterine cavity required neovascularization (Taylor, et al. (2002) Ann. NY Acad. Sci. 955:89-100). In atherosclerosis, the main cause of heart attack, the growth of atherosclerotic plaques within the coronary artery is dependent on angiogenesis (Moulton, et al. (2001) Curr. Atheroscler. Rep. 3:225-233). It is a complex series of interactions between the expanding tissues or lesions and their host microenvironment that trigger stimulation of angiogenesis in these pathological conditions. The surrounding stroma, and infiltrating blood-derived cells (e.g., macrophages, mast cells, T-cells, monocytes, leukocytes, and platelets) are known sources of pro-angiogenesis factors. However, in many cases, the cells of the aberrantly growing tissues (e.g., cancer cells) themselves produce pro-angiogenic factors (Carmeliet & Jain (2000) supra).

Among many factors known to promote angiogenesis, VEGF (vascular endothelial growth factor) is one of the most potent pro-angiogenic factors frequently upregulated in these pathologies (Celletti, et al. (2001) Nat. Med. 7:425-429; Inoue, et al. (1998) Circulation 98:2108-2116; Donnez, et al. (1998) Hum. Reprod. 13:1686-1690; Carmeliet & Jain (2000) supra; Ferrara (1999) J. Mol. Med. 77:527-543. The elevated production of VEGF can be triggered by limited oxygen, i.e., hypoxia, found within the microenvironment of growing tissues or lesions.

As noted above, it has been observed that fibroblast cells expressing a RTK oncoprotein engineered to recruit only the Grb2 or Shc adaptor proteins show similar transforming activities in tissue culture assays (Saucier, et al. (2002) supra). However, as described herein, when injected subcutaneously in nude mice, cells expressing RTK oncoproteins that recruit the Shc adaptor protein formed tumors with a short latency (˜7 days) and expanded rapidly. The short latency of tumor formation correlated with the capacity of these cells to produce VEGF in their culture media and to induce a robust angiogenic response in mice when seeded in MATRIGEL. In contrast, cells expressing RTK oncoproteins that recruit the Grb2 adaptor protein induced tumors that grew rapidly, but only after a prolonged latency (˜24 days) (Table 1). These cells were transformed, but unable to produce VEGF and were devoid of angiogenic properties in an in vivo MATRIGEL angiogenesis assay. The ability of Shc binding RTK oncoproteins to induce VEGF production was not dependent on the constitutive activity of these oncoproteins. Substitution of the Grb2 or Shc binding sites into a null signaling mutant of the Met receptor (CSF-Met Y1349/1356F), demonstrated that VEGF production was induced following ligand stimulation of the Shc binding RTK variant, but not downstream from the Grb2 binding RTK variant. The importance of Shc recruitment to RTKs for the induction of VEGF production was demonstrated downstream of another RTK family member, the Neu/ErbB-2/HER2 RTK. The production of VEGF was increased downstream of an activated Neu/ErbB-2 add-back RTK mutant in which only the Shc binding site was reintroduced, but not by an Neu/ErbB2 add-back RTK mutant binding to Grb2.

Overall, the findings herein indicate that recruitment of Grb2 or Shc adaptor proteins to RTKs is not functionally redundant. While the recruitment of Grb2 to an activated RTK promotes cell transformation and tumorigenesis, the binding of Shc to a RTK not only induces cell transformation, but in addition confers an intrinsic capacity for cells to induce angiogenesis, at least in part through an upregulation of VEGF protein. The ability of Shc to induce angiogenesis provides an advantage for the early onset of tumorigenesis induced by the Shc binding RTK variants, by promoting the early initiation of tumor vascularization.

A requirement for Shc in the enhanced VEGF production downstream from RTKs was further established using Shc null fibroblasts. In contrast to wild-type MEF cells, a Tpr-Met RTK oncoprotein was unable to induce VEGF production in MEF cells derived from ShcA-deficient mice. Importantly the induction of VEGF by Tpr-Met was rescued by complementation with the ShcA gene. These results demonstrate that Shc-dependent signaling pathway(s) is/are essential for VEGF induction by the Met receptor oncoprotein.

The mechanisms regulating VEGF expression are complex and vary depending on the cell context and the receptor investigated, including enhanced stability of VEGF mRNA, as well as transcriptional activation of the VEGF gene. The results described herein show that the enhanced production of VEGF protein induced by the Shc binding variants, or the Tpr-Met oncoprotein, correlated with an increase in the level of VEGF mRNA.

Further, it was found that Shc stimulates angiogenesis by modulating the expression of other modulators of angiogenesis, including downregulating the expression of thrombospondin-1 (TPS-1) and angiopoietin-1 (Ang-1), as well as increasing the expression of fibroblast growth factor-2 (FGF-2) and Angiopoietin-2 (Ang-2).

FGF-2, also known as basic FGF, is a powerful stimulator of angiogenesis that induces the proliferation, migration, differentiation, or survival of endothelial cells, and supports growth of cells such as smooth muscle cells and pericytes (Bikfalvi, et al. (1997) Endocr. Rev. 18:26-45; Friesel and Maciag (1995) Faseb J. 9:919-925; Klein, et al. (1997) EXS 79:159-192; Slavin (1995) Cell Biol. Int. 19:431-444). TSP-1 is the first endogenous angiogenesis inhibitor to be identified (Good, et al. (1990) Proc. Natl. Acad. Sci. USA 97:6624-6628). TSP-1 inhibits angiogenesis by reducing the activity of the MMP-9 (Rodriguez-Manzaneque, et al. (2001) Proc. Natl. Acad. Sci. USA 98:12485-12490), a matrix metalloproteinase which promotes the association of VEGF with its receptor by releasing VEGF from the extracellular matrix (Ribatti, et al. (1998) Int. J. Cancer 77:449-454). Angiopoietins play important roles in angiogenesis by controlling the maturation and stabilization of blood vessels (Yancopoulos, et al. (2000) Nature (London) 407:242-248). Ang-1 and Ang-2 has been identified, respectively, as agonist and antagonist of the Tie2 receptor signaling (Maisonpierre, et al. (1997) Science 277:55-60; Suri, et al. (1996) Cell 87:1171-1180). Ang-1 promotes stabilization of blood vessels by inducing the recruitment and maintenance of an association between peri-endothelial supporting cells and endothelial cells (Suri, et al. (1996) supra; Suri, et al. (1998) Science 282:468-471), whereas Ang-2 counteracts the effect of Ang-1 (Maisonpierre, et al. (1997) supra) and promotes the regression of blood in the absence of endothelial survival factors such as VEGF or FGF-2 (Holash, et al. (1999) Science 284:1994-1998; Holash, et al. (1999) Oncogene 18:5356-5362). However, in the presence of VEGF or FGF-2, the block by Ang-2 of the stabilizing effect of Ang-1 on new vessel sprouting, cooperates to induce blood vessel growth by enhancing vessel plasticity and thus the responsiveness to VEGF-mediated neovascularization (Holash, et al. (1999) supra; Holash, et al. (1999) supra; Koga, et al. (2001) Cancer Res. 61:6248-6254).

Therefore, the results provided herein demonstrate that the activation of Shc-dependent signaling pathways induces angiogenesis by tipping the balance of pro- and anti-angiogenic factors, including VEGF, FGF-2, TSP-1, as well as Ang-1 and Ang-2, which is in favor of pro-angiogenesis.

Accordingly, the invention provides methods and materials for modulating angiogenesis, VEGF production and expression of modulators of angiogenesis based on the modulation of Shc expression and activity. The invention further provides methods and materials for the preventing or treating an angiogenesis-related disease or process via modulating the activity or expression of Shc.

As used herein, angiogenesis refers to the generation of new blood vessels in a tissue or organ. This process occurs in animals under normal physiological conditions in certain situations, e.g., during wound healing, fetal and embryonic development, and the formation of other tissues such as the corpus luteum, endometrium and placenta. Abnormal angiogenesis, i.e., either greater or less than normal levels depending on the tissue and situation, has been in some cases related to disease, herein referred to as an angiogenesis-related disease or process, which, as used herein, refers to a disease or process in which the level of angiogenesis, either directly or indirectly, contributes to disease onset and/or progression.

One aspect of the present invention is a method for modulating angiogenesis in a cell, tissue, or subject (e.g., a mammal such as a human) by contacting a cell, tissue, or subject with an agent which regulates the expression or activity of Shc. The agent can interact directly with Shc or the coding sequence for Shc to modulate the activity thereof. Alternatively, the agent can interact with any other polypeptide, nucleic acid or other molecule if such interaction results in a modulation of Shc activity. As disclosed herein, by regulating the expression or activity of Shc, the production of VEGF, or expression of modulators of angiogenesis (i.e., FGF-2, TSP-1, Ang-1 or Ang-2) is altered. In one embodiment, increasing the expression or activity of Shc results in an increase in VEGF production, an increase in the expression of angiogenesis modulators such as FGF-2 or Ang-2 or a decrease in the expression of angiogenesis modulators such as Tsp-1 or Ang-1 thereby promoting angiogenesis in the cell, tissue or subject. In another embodiment, decreasing the expression or activity of Shc results in a decrease in VEGF production, a decrease in the expression of angiogenesis modulators such as FGF-2 or Ang-2 or an increase in the expression of angiogenesis modulators such as Tsp-1 or Ang-1 thereby inhibiting angiogenesis in the cell, tissue, or subject.

As Shc regulates the expression of multiple modulators of angiogenesis (i.e. VEGF, Tsp-1 Ang-1, Ang-2 and FGF-2), it is contemplated that Shc may be a key regulator of a plurality of pro-angiogenic factors including IL-8, EGF, Transforming Growth Factor-Beta (TGF-β), Tumor Necrosis Factor (TNF), Platelet Derived Growth Factor, and Placental growth factor (PLGF), as well as anti-angiogenic factors including, Chondromodulin-I (ChM-I), pigment epithelium-derived factor (PEDF), angiostatin, endostatin, interferons, interleukin, and platelet factor 4 or other modulators of angiogenesis such as matrix metalloproteinase-2 (MMP-2), matrix metalloproteinase-7 (MMP-7), matrix metalloproteinase-9 (MMP-9) or CD44. The effect of Shc on the expression of these modulators can be determined in knockout or knockdown experiments and by northern blot analysis or RT-PCR as disclosed herein or by microarray analysis. Furthermore, post-translational processing of these modulators can be carried out using standard protein analysis methods such as SDS-PAGE and immunoblot analysis or proteomic approaches.

Agents of this invention can enhance or increase, or inhibit or decrease the activity or expression of Shc, and can further be an Shc inactivator or an Shc activator. The term Shc activator, as used herein, refers to a molecule that directly binds to Shc or a nucleic acid encoding Shc to increase or enhance the activity or expression thereof. The term Shc inactivator, as used herein, refers to a molecule that directly binds to Shc or a nucleic acid encoding Shc to inhibit or reduce the activity or expression thereof.

The term agent, as used herein, is intended to be interpreted broadly and encompasses organic and inorganic molecules. Organic compounds include, but are not limited to polypeptides, lipids, carbohydrates, coenzymes and nucleic acid molecules. Polypeptides include but are not limited to antibodies and enzymes. Nucleic acids include but are not limited to DNA, RNA and DNA-RNA chimeric molecules. Suitable RNA molecules include RNAi, antisense RNA molecules and ribozymes. The nucleic acid can further encode any polypeptide such that administration of the nucleic acid and production of the polypeptide results in a modulation of Shc activity.

A wide variety of alternative genomic approaches are available for administration of nucleic acids that encode Shc to a subject for therapeutic or other purposes. For example, in one embodiment, transformation of cells with antisense constructs can be used to inhibit expression of Shc. The coding sequences for Shc from a variety of species are known and an antisense nucleotide sequence or nucleic acid encoding an antisense nucleotide sequence can be generated to any portion thereof in accordance with known techniques.

The term antisense nucleotide sequence, as used herein, refers to a nucleotide sequence that is complementary to a specified DNA or RNA sequence. Antisense RNA sequences and nucleic acids that express the same can be made in accordance with conventional techniques. See, e.g., U.S. Pat. Nos. 5,023,243 and 5,149,797.

An antisense nucleotide sequence that can be used to carry out the invention is a nucleotide sequence that is complementary to the nucleotide sequences including, but are not limited to, Human Shc DNA sequence (SEQ ID NO:1, Accession no. X68148.1, Pelicci, et al. (1992) Cell 70(1):93-104); Human Shc1 DNA sequence (SEQ ID NO:3, Accession no. NM_{—}003029.1, Pelicci, et al. (1992) supra, Huebner, et al. (1994) Genomics 22(2):281-287, Migliaccio, et al. (1997) EMBO J. 16(4):706-716); Human p66 Shc DNA sequence (SEQ ID NO:5, Accession no. Y09847.1, Harun, et al. (1997) Genomics 42(2):349-352); Mouse Shc1 DNA sequence (SEQ ID NO:7, Accession no. NM_{—}011368, Blaikie, et al (1994) J. Biol. Chem. 269(51):32031-32034); Mouse p66Shc DNA sequence (SEQ ID NO:9, Accession no. U46956.2), or portions thereof. An antisense nucleotide sequence can be designed that is specific for, for example, human Shc by directing the antisense nucleotide sequence to the human sequences (e.g., SEQ ID NO:1, SEQ ID NO:3, SEQ ID NO:5) or homologues thereof.

Homology, homologous, or homologue refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is homologous to another sequence if the two sequences are substantially identical and the functional activity of the sequences is conserved. Two nucleic acid sequences are considered substantially identical if, when optimally aligned (with gaps permitted), they share at least about 50% sequence similarity or identity, or if the sequences share defined functional motifs. In alternative embodiments, sequence similarity in optimally aligned substantially identical sequences can be at least 60%, 70%, 75%, 80%, 85%, 90% or 95%. As used herein, a given percentage of homology between sequences denotes the degree of sequence identity in optimally aligned sequences. An unrelated or non-homologous sequence shares less than 40% identity, or less than about 25% identity, with any of nucleic acid sequences of the invention (i. e., SEQ ID NO:1, SEQ ID NO: 3, SEQ ID NO: 5, SEQ ID NO:7, SEQ ID NO:9) as well as the protein sequences of the invention (SEQ ID NO:2, SEQ ID NO:4, SEQ ID NO:6, SEQ ID NO:8, SEQ ID NO:10).

Substantially complementary nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, or at least 95%. Optimal alignment of sequences for comparisons of identity can be conducted using a variety of algorithms, such as the local homology algorithm of Smith and Waterman ((1981) Adv. Appl. Math 2:482), the homology alignment algorithm of Needleman and Wunsch ((1970) J. Mol. Biol. 48:443), the search for similarity method of Pearson and Lipman ((1988) Proc. Natl. Acad. Sci. USA 85: 2444), and the computerized implementations of these algorithms (such as GAP, BESTFIT, FASTA and TFASTA in the Wisconsin Genetics Software Package, Genetics Computer Group, Madison, Wis.). Sequence identity may also be determined using the BLAST algorithm, described in Altschul, et al. ((1990) J. Mol. Biol. 215:403-10) (using the published default settings). Software for performing BLAST analysis is available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff (1992) Proc. Natl. Acad. Sci. USA 89:10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, less than about 0.1, less than about 0.01, or less than about 0.001.

An alternative indication that two nucleic acid sequences are substantially complementary is that the two sequences hybridize to each other under moderately stringent or stringent conditions as described herein.

Those skilled in the art will appreciate that it is not necessary that the antisense nucleotide sequence be fully complementary to the target sequence as long as the degree of sequence similarity is sufficient for the antisense nucleotide sequence to hybridize to its target and reduce production of Shc polypeptide (e.g., by at least about 40%, 50%, 60%, 70%, 80%, 90%, 95% or more). As is known in the art, a higher degree of sequence similarity is generally required for short antisense nucleotide sequences, whereas a greater degree of mismatched bases will be tolerated by longer antisense nucleotide sequences.

In representative embodiments of the invention, the antisense nucleotide sequence will hybridize to the nucleotide sequences encoding Shc specifically disclosed herein (e.g., SEQ ID NO:1, SEQ ID NO:3, SEQ ID NO:5, SEQ ID NO:7 or SEQ ID NO:9 or portions thereof) and will reduce the level of Shc polypeptide.

For example, hybridization of such nucleotide sequences can be carried out under conditions of reduced stringency, medium stringency or even stringent conditions (e.g., conditions represented by a wash stringency of 35-40% Formamide with 5× Denhardt's solution, 0.5% SDS and 1× SSPE at 37° C.; conditions represented by a wash stringency of 40-45% Formamide with 5× Denhardt's solution, 0.5% SDS, and 1× SSPE at 42° C.; and/or conditions represented by a wash stringency of 50% Formamide with 5× Denhardt's solution, 0.5% SDS and 1× SSPE at 42° C., respectively) to the nucleotide sequences specifically disclosed herein. See, e.g., Sambrook et al., Molecular Cloning, A Laboratory Manual (2d Ed. 1989) (Cold Spring Harbor Laboratory).

Alternatively stated, antisense nucleotide sequences of the invention have at least about 60%, 70%, 80%, 90%, 95%, 97%, 98% or higher sequence similarity with the complement of the Shc coding sequences specifically disclosed herein and will reduce the level of Shc polypeptide production.

In other embodiments, the antisense nucleotide sequence can be directed against any coding sequence, the silencing of which results in a modulation of Shc activity.

The length of the antisense nucleotide sequence (i.e., the number of nucleotides therein) is not critical as long as it binds selectively to the intended location and reduces transcription and/or translation of the target sequence (e.g., by at least about 40%, 50%, 60%, 70%, 80%, 90%, 95% or more), and can be determined in accordance with routine procedures. In general, the antisense nucleotide sequence will be from about eight, ten or twelve nucleotides in length up to about 20, 30, 50, 60 or 70 nucleotides, or longer, in length.

In another embodiment, RNA interference (RNAi) is used to modulate Shc activity. The RNAi can be directed against the Shc coding sequence in the cell or any other sequence that results in modulation of Shc activity.

RNAi is a mechanism of post-transcriptional gene silencing in which double-stranded RNA (dsRNA) corresponding to a coding sequence of interest is introduced into a cell or an organism, resulting in degradation of the corresponding mRNA. The RNAi effect persists for multiple cell divisions before gene expression is regained. RNAi is therefore a powerful method for making targeted knockouts or knockdowns at the RNA level. RNAi has proven successful in human cells, including human embryonic kidney and HeLa cells (see, e.g., Elbashir, et al. (2001) Nature 411:494-8). In one embodiment, silencing can be induced in mammalian cells by enforcing endogenous expression of RNA hairpins (see, Paddison, et al. (2002) PNAS USA 99:1443-1448). In another embodiment, transfection of small (e.g., 21-23 nucleotide) dsRNA specifically inhibits nucleic acid expression (reviewed in Caplen (2002) Trends Biotech. 20:49-51).

The mechanism by which RNAi achieves gene silencing has been reviewed in Sharp, et al. (2001) Genes Dev 15:485-490; and Hammond, et al. (2001) Nature Rev. Gen. 2:110-119).

RNAi technology utilizes standard molecular biology methods. RNAi may be effected by the introduction of suitable in vitro synthesized siRNA or siRNA-like molecules into cells. RNAi may for example be performed using chemically-synthesized RNA. Alternatively, suitable expression vectors can be used to transcribe such RNA either in vitro or in vivo. In vitro transcription of sense and antisense strands (encoded by sequences present on the same vector or on separate vectors) can be effected using for example T7 RNA polymerase, in which case the vector can contain a suitable coding sequence operably-linked to a T7 promoter. The in vitro-transcribed RNA can in embodiments be processed (e.g., using E. coli RNase III) in vitro to a size conducive to RNAi. The sense and antisense transcripts are combined to form an RNA duplex which is introduced into a target cell of interest. Other vectors can be used, which express small hairpin RNAs (shRNAs) which can be processed into siRNA-like molecules. Various vector-based methods are described in for example Brummelkamp, et al. (2002) Science 296(5567):550-3; Lee, et al. (2002) Nat. Biotechnol. 20(5):500-5; Miyagashi and Taira (2002) Nat. Biotechnol. 20(5):497-500; Paddison, et al. (2002) Proc. Natl. Acad. Sci. USA 99(3):1443-8; Paul, et al. (2002); and Sui, et al. (2002) Proc. Natl. Acad. Sci. USA 99(8):5515-20. Various methods for introducing such vectors into cells, either in vitro or in vivo (e.g., gene therapy) are known in the art.

Kits for production of dsRNA for use in RNAi are available commercially, e.g., from New England Biolabs, Inc. and Ambion Inc. (Austin, Tex., USA). Methods of transfection of dsRNA or plasmids engineered to make dsRNA are routine in the art.

Accordingly, in one embodiment, Shc expression can be inhibited by introducing into or generating within a cell an siRNA or siRNA-like molecule corresponding to a Shc-encoding nucleic acid or fragment thereof, or to an nucleic acid homologous thereto. An siRNA-like molecule refers to a nucleic acid molecule similar to an siRNA (e.g., in size and structure) and capable of eliciting siRNA activity, i.e., to effect the RNAi-mediated inhibition of expression. In various embodiments, such a method can entail the direct administration of the siRNA or siRNA-like molecule into a cell, or use of the vector-based methods described herein. In an embodiment, the siRNA or siRNA-like molecule is less than about 30 nucleotides in length. In a further embodiment, the siRNA or siRNA-like molecule is about 21-23 nucleotides in length. In another embodiment, an siRNA or siRNA-like molecule is a 19-21 bp duplex portion, each strand having a two nucleotide 3′ overhang. In particular embodiments, the siRNA or siRNA-like molecule is substantially identical to an Shc-encoding nucleic acid or a fragment or variant (or a fragment of a variant) thereof. Such a variant is capable of encoding a protein having Shc-like activity. In other embodiments, the sense strand of the siRNA or siRNA-like molecule is substantially identical to SEQ ID NO:1, SEQ ID NO:3, SEQ ID NO:5, SEQ ID NO:7, or SEQ ID NO:9, or a fragment thereof (RNA having U in place of T residues of the DNA sequence).

Silencing effects similar to those produced by RNAi have been reported in mammalian cells with transfection of a mRNA-cDNA hybrid construct (Lin, et al. (2001) Biochem. Biophys. Res. Commun. 281:639-44), providing yet another strategy for silencing a coding sequence of interest.

In a further embodiment, the agent can further by a ribozyme. Ribozymes are RNA-protein complexes that cleave nucleic acids in a site-specific fashion. Ribozymes have specific catalytic domains that possess endonuclease activity (Kim, et al. (1987) Proc. Natl. Acad. Sci. USA 84:8788; Gerlach, et al. (1987) Nature 328:802; Forster and Symons (1987) Cell 49:211). For example, a large number of ribozymes accelerate phosphoester transfer reactions with a high degree of specificity, often cleaving only one of several phosphoesters in an oligonucleotide substrate (Michel and Westhof (1990) J. Mol. Biol. 216:585; Reinhold-Hurek and Shub (1992) Nature 357:173). This specificity has been attributed to the requirement that the substrate binds via specific base-pairing interactions to the internal guide sequence (IGS) of the ribozyme prior to chemical reaction.

Ribozyme catalysis has primarily been observed as part of sequence-specific cleavage/ligation reactions involving nucleic acids (Joyce (1989) Nature 338:217). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon, et al. (1991) Proc. Natl. Acad. Sci. USA 88:10591; Sarver, et al. (1990) Science 247:1222; Sioud, et al. (1992) J. Mol. Biol. 223:831).

Therefore, in alternative embodiments, the invention provides antisense molecules, siRNA or siRNA-like molecules, and ribozymes for exogenous administration to effect the degradation or inhibition of the translation of Shc mRNA. Examples of therapeutic antisense oligonucleotide applications, incorporated herein by reference, include: U.S. Pat. Nos. 5,135,917; 5,098,890; 5,087,617; 5,166,195; 5,004,810; 5,194,428; 4,806,463; 5,286,717; 5,276,019 and 5,264,423.

Nucleic acid molecules of the present invention have a sufficient degree of complementarity to the Shc mRNA to avoid non-specific binding of the nucleic acid molecule to non-target sequences under conditions in which specific binding is desired, such as under physiological conditions in the case of in vivo assays or therapeutic treatment or, in the case of in vitro assays, under conditions in which the assays are conducted. The target mRNA for nucleic acid molecule binding can include not only the information to encode a protein, but also associated ribonucleotides, which for example form the 5′-untranslated region, the 3′-untranslated region, the 5′ cap region and intron/exon junction ribonucleotides. A method of screening for antisense, siRNA and ribozyme nucleic acids that can be used to provide such molecules as Shc inhibitors of the invention is disclosed in U.S. Pat. No. 5,932,435 (which is incorporated herein by reference).

Nucleic acid molecules (oligonucleotides) of the invention can include those which contain intersugar backbone linkages such as phosphotriesters, methyl phosphonates, short chain alkyl or cycloalkyl intersugar linkages or short chain heteroatomic or heterocyclic intersugar linkages, phosphorothioates and those with CH₂—NH—O—CH₂, CH₂—N(CH₃)—O—CH₂ (known as methylene(methylimino) or MMI backbone), CH₂—O—N(CH₃)—CH₂, CH₂—N(CH₃)—N(CH₃)—CH₂ and O—N(CH₃)—CH₂ —CH₂ backbones (where phosphodiester is O—P—O—CH₂). Oligonucleotides having morpholino backbone structures cam also be used (U.S. Pat. No. 5,034,506). In alternative embodiments, oligonucleotides can have a peptide nucleic acid (PNA, sometimes referred to as protein nucleic acid) backbone, in which the phosphodiester backbone of the oligonucleotide is replaced with a polyamide backbone wherein nucleosidic bases are bound directly or indirectly to aza nitrogen atoms or methylene groups in the polyamide backbone (Nielsen, et al. (1991) Science 254:1497 and U.S. Pat. No. 5,539,082). The phosphodiester bonds can be substituted with structures which are chiral and enantiomerically specific. Persons of ordinary skill in the art will be able to select other linkages for use in practice of the invention.

Oligonucleotides can also include species which include at least one modified nucleotide base. Thus, purines and pyrimidines other than those normally found in nature can be used. Similarly, modifications on the pentofuranosyl portion of the nucleotide subunits can also be effected. Examples of such modifications are 2′-O-alkyl- and 2′-halogen-substituted nucleotides. Some specific examples of modifications at the 2′ position of sugar moieties which are useful in the present invention are OH, SH, SCH₃, F, OCN, O(CH₂)ₙ NH₂ or O(CH₂)ₙ CH₃ where n is from 1 to about 10; C₁ to C₁₀ lower alkyl, substituted lower alkyl, alkaryl or aralkyl; Cl; Br; CN; CF₃; OCF₃; O—, S—, or N-alkyl; O—, S—, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. One or more pentofuranosyl groups can be replaced by another sugar, by a sugar mimic such as cyclobutyl or by another moiety which takes the place of the sugar.

In some embodiments, the oligonucleotides in accordance with this invention can be from about 5 to about 100 nucleotide units. As will be appreciated, a nucleotide unit is a base-sugar combination (or a combination of analogous structures) suitably bound to an adjacent nucleotide unit through phosphodiester or other bonds forming a backbone structure.

In yet a further embodiment, an agent of the invention can be an antibody or antibody fragment. The antibody or antibody fragment can bind to Shc resulting in modulation of Shc activity (e.g., as an agonist or antagonist). By way of illustration, an antibody of the present invention binds to a Shc protein of SEQ ID NO:2, SEQ ID NO:4, SEQ ID NO:6, SEQ ID NO:8, or SEQ ID NO:10.

The term antibody or antibodies as used herein refers to all types of immunoglobulins, including IgG, IgM, IgA, IgD, and IgE. The antibody can be monoclonal or polyclonal and can be of any species of origin, including (for example) mouse, rat, rabbit, horse, or human, or can be a chimeric antibody. See, e.g., Walker, et al. (1989) Mol. Immunol. 26:403-11. The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980.

Antibody fragments included within the scope of the present invention include, for example, Fab, F(ab′)₂, and Fc fragments, and the corresponding fragments obtained from antibodies other than IgG. Such fragments can be produced by known techniques. For example, F(ab′)₂ fragments can be produced by pepsin digestion of the antibody molecule, and Fab fragments can be generated by reducing the disulfide bridges of the F(ab′)₂ fragments. Alternatively, Fab expression libraries can be constructed to allow rapid and easy identification of monoclonal Fab fragments with the desired specificity (Huse, et al. (1989) Science 254:1275-1281).

Polyclonal antibodies used to carry out the present invention can be produced by immunizing a suitable animal (e.g., rabbit, goat, etc.) with an antigen to which a monoclonal antibody to the target binds, collecting immune serum from the animal, and separating the polyclonal antibodies from the immune serum, in accordance with known procedures.

Monoclonal antibodies used to carry out the present invention can be produced in a hybridoma cell line according to the technique of Kohler and Milstein (1975) Nature 265:495-97. For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in E. coli by recombinant techniques known to those skilled in the art. See, e.g., Huse (1989) Science 246:1275-81.

Antibodies specific to the target polypeptide can also be obtained by phage display techniques known in the art.

In particular embodiments of the invention, various means for reducing or decreasing the expression or activity of Shc are provided for inhibiting angiogenesis. In alternative embodiments, means for increasing the expression or activity of Shc are provided for stimulating angiogenesis. For example, Shc expression can be increased by introducing into or generating within a cell a recombinant Shc protein molecule or functional fragment thereof. Expression vectors and methods for introducing said expression vectors into cells are provided herein.

Another aspect of the present invention relates to the use of an Shc as a target in screening assays that can be used to identify agents that are useful for the prevention or treatment of an angiogenesis-related disease or process.

In one embodiment, such an assay involves the steps of contacting Shc with a test agent and measuring Shc activity in the presence and absence of the test agent, wherein a lower measured activity in the presence of the test agent, as compared to the measured activity in the absence of the test agent, indicates that the agent is an inhibitor of an Shc-dependent signal and is useful for the inhibiting angiogenesis. Conversely, an elevated measured activity in the presence of the test agent, as compared to the measured activity in the absence of the test agent, indicates that the agent is an activator of an Shc-dependent signal and is useful for the stimulating angiogenesis. Activators and inhibitors of Shc activity are useful in the prevention or treatment of an angiogenesis-related disease or process.

Shc activity, as used herein, refers to any type of observed phenomenon which can be attributed to Shc, via for example the study of phosphorylation of Shc tyrosine residues (e.g., Y239/240 and/or Y317) or via the study of Shc binding to its binding partners, e.g., RTKs or Grb2, as well as via the study of phenomena related to and downstream to Shc-dependent signals, such as the activation of downstream cellular products (e.g., changes in phosphorylation; changes in enzymatic activity and the levels of gene expression, gene products and second messengers) and processes (e.g,. cellular transformation, cell migration, angiogenesis).

For example, a binding assay of the invention involves the steps of contacting an Shc with a test agent in the presence of a binding partner for Shc and assaying the binding activity of the Shc with binding partner in the presence and the absence of the test agent, to identify agents that inhibit or stimulate Shc binding, wherein said agent is useful for the prevention or treatment of angiogenesis-related disease or process. In particular embodiments, the binding partner is RTK and Grb2. In other embodiments, the RTK is Met, Tpr-Met and the Y-Shc-1 and Y-Shc-2 binding variants, and the RTK-Shc (modified CSF-Met chimera mutant) binding variants, and Neu/ErbB2 and Neu/Erb2 add-back mutant (Shc Neu/ErbB2 YD add-back) described herein. In further embodiments, the binding is mediated by the PTB, phosphotyrosine and/or SH2 regions of Shc.

The assay methods of the invention can further be used to identify agents capable of modulating (e.g., inhibiting or stimulating) angiogenesis in a biological system. Such an assay can further involve the step of assaying the agent for the reduction, abrogation or reversal of angiogenesis as well as the stimulation or promotion of angiogenesis. A number of assays for angiogenesis can be used, such as the MATRIGEL assay described herein. In particular embodiments, the above noted biological system can be a mammal, such as a human, or a suitable animal model system such as a rodent (e.g., mouse). A biological system, as used herein, refers to any system (either in vitro or in vivo) encompassing biological material, such as, for example, a cell or cells, culture, tissue, organism, animal, etc.

Screening assays of the invention can also be utilized to identify or characterize an agent for modulating (e.g., inhibiting or stimulating) angiogenesis. Therefore, the invention further provides a method for identifying or characterizing an agent for regulating production of modulators of angiogenesis, said method involves contacting a first cell expressing an Shc with a test agent and measuring the production of a modulator of angiogenesis in said first cell as compared to a second cell expressing Shc which has not been contacted with the test agent, wherein a higher measured production of pro-angiogenic factor production and lower measured production of anti-angiogenic factor production of said first cell compared to said second cell is indicative that the test agent is useful for stimulating angiogenesis. Conversely, a higher measured production of anti-angiogenic factor production and lower measured production of pro-angiogenic factor production of said first cell compared to said second cell is indicative that the test agent is useful for inhibiting angiogenesis.

Such gene production or expression can be measured by detection of the corresponding RNA or protein, or via the use of a suitable reporter construct comprising a transcriptional regulatory element(s) normally associated with such a modulator of angiogenesis gene, operably-linked to a reporter gene. A first nucleic acid sequence is operably-linked with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since, for example, enhancers generally function when separated from the promoters by several kilobases and intronic sequences can be of variable lengths, some polynucleotide elements can be operably-linked but not contiguous. Transcriptional regulatory element is a generic term that refers to DNA sequences, such as initiation and termination signals, enhancers, and promoters, splicing signals, polyadenylation signals which induce or control transcription of protein coding sequences with which they are operably-linked. The expression of such a reporter gene can be measured on the transcriptional or translational level, e.g., by the amount of RNA or protein produced. RNA can be detected by, for example, northern analysis or by the reverse transcriptase-polymerase chain. reaction (RT-PCR) method (see, for example, Sambrook, et al. (1989) Molecular Cloning: A Laboratory Manual (second edition), Cold Spring Harbor Laboratory Press, Cold Spring Harbor, N.Y., USA). Protein levels can be detected either directly using affinity reagents (e.g., an antibody or fragment thereof using methods such as described in Harlow and Lane (1988) Antibodies: A Laboratory Manual, Cold Spring Harbor Laboratory Press, Cold Spring Harbor, N.Y. or a ligand which binds the protein) or by other properties (e.g., fluorescence in the case of green fluorescent protein) or by measurement of the protein's activity, which can entail enzymatic activity to produce a detectable product (e.g., with altered spectroscopic properties) or a detectable phenotype (e.g., alterations in cell growth). Suitable reporter genes include, but are not limited to, chloramphenicol acetyltransferase, beta-D galactosidase, luciferase, or green fluorescent protein. It is contemplated that microarray technology can be used to carry out this assay of the invention, as more than one modulator of angiogenesis can be analyzed.

The above-noted methods and assays can be employed either with a single test agent or a plurality or library (e.g., a combinatorial library) of test agents. In the latter case, synergistic effects provided by combinations of agents can also be identified and characterized. The above-mentioned agents can be used for inhibiting or stimulating angiogenesis, VEGF production, expression of a modulator of angiogenesis and for the prevention or treatment of an angiogenesis-related disease or process, or may be used as lead compounds for the development and testing of additional compounds having improved specificity, efficacy or pharmacological (e.g., pharmacokinetic) properties. In certain embodiments, one or a plurality of the steps of the screening/testing methods of the invention can be automated.

Such assay systems can involve a variety of means to enable and optimize useful assay conditions. Such means can include, but are not limited to, suitable buffer solutions, for example, for the control of pH and ionic strength and to provide any necessary components for optimal Shc activity and stability (e.g., protease inhibitors), temperature control means for optimal Shc activity and or stability, and detection means to enable the detection of the Shc activity. A variety of such detection means can be used including, but not limited to, one or a combination of the following: radiolabelling (e.g., ³²P), antibody-based detection, fluorescence, chemiluminescence, spectroscopic methods (e.g., generation of a product with altered spectroscopic properties), various reporter enzymes or proteins (e.g., horseradish peroxidase, green fluorescent protein), specific binding reagents (e.g., biotin/(streptavidin), and others. Binding can also be analyzed using generally known methods in this area, such as electrophoresis on native polyacrylamide gels, as well as fusion protein-based assays such as the yeast 2-hybrid system or in vitro association assays, or proteomics-based approaches to identify Shc binding proteins.

Assays can be carried out in vitro utilizing a source of Shc which is naturally isolated or recombinantly produced Shc, in preparations ranging from crude to pure. Recombinant Shc can be produced in a number of prokaryotic or eukaryotic expression systems which are well-known in the art. Such assays can be performed in an array format. In certain embodiments, one or a plurality of the assay steps are automated.

A homolog, variant or fragment of Shc which retains activity can also be used in the methods of the invention. Homologs include protein sequences which are substantially identical to the amino acid sequence of an Shc, sharing significant structural and functional homology with an Shc. Variants include, but are not limited to, proteins or peptides which differ from an Shc by any modifications, or amino acid substitutions, deletions or additions. Such variants include fusion proteins, for example, a protein of interest or portion thereof fused with a suitable fusion domain (such as glutathione-S-transferase fusions and others). Modifications can occur anywhere including the polypeptide backbone (i.e., the amino acid sequence), the amino acid side chains and the amino or carboxy termini. Such substitutions, deletions or additions can involve one or more amino acids. Fragments include a fragment or a portion of a Shc or a fragment or a portion of a homolog or variant of a Shc.

Assays can, in an embodiment, be performed using an appropriate host cell as a source of Shc. Such a host cell can be prepared by the introduction of DNA encoding Shc into the host cell and providing conditions for the expression of Shc. Such host cells can be prokaryotic or eukaryotic, bacterial, yeast, amphibian or mammalian.

Nucleic acids (e.g., for overexpression of Shc for therapy or assays of the invention, or to effect antisense or RNAi-based methods) may be delivered to cells in vivo using methods such as direct injection of DNA, receptor-mediated DNA uptake, viral-mediated transfection or non-viral transfection and lipid based transfection, all of which may involve the use of gene therapy vectors. Direct injection has been used to introduce naked DNA into cells in vivo (see, e.g., Acsadi, et al. (1991) Nature 332:815-818; Wolff, et al. (1990) Science 247:1465-1468). A delivery apparatus (e.g., a gene gun) for injecting DNA into cells in vivo can be used. Such an apparatus is commercially available (e.g., from BioRad). Naked DNA can also be introduced into cells by complexing the DNA to a cation, such as polylysine, which is coupled to a ligand for a cell-surface receptor (see, for example, Wu and Wu (1988) J. Biol. Chem. 263:14621; Wilson, et al. (1992) J. Biol. Chem. 267:963-967; and U.S. Pat. No. 5,166,320). Binding of the DNA-ligand complex to the receptor can facilitate uptake of the DNA by receptor-mediated endocytosis. A DNA-ligand complex linked to adenovirus capsids which disrupt endosomes, thereby releasing material into the cytoplasm, can be used to avoid degradation of the complex by intracellular lysosomes (see, for example, Curiel,et al. (1991) Proc. Natl. Acad. Sci. USA 88:8850; Cristiano, et al. (1993) Proc. Natl. Acad. Sci. USA 90:2122-2126).

It is further contemplated that Shc may be administered via stem cells which are genetically engineered to produce Shc.

Cells to be targeted by nucleic acid molecules of the invention include, but are not limited to, an endothelial cell, a lymphocyte, a macrophage, a glia cell, a fibroblast, a liver cell, a kidney cell, a muscle cell, a cell of the bone or cartilage tissue, a synovial cell, a peritoneal cell, a skin cell, an epithelial cell, a leukemia cell or a tumor cell.

Defective retroviruses are well-characterized for use as gene therapy vectors (see Miller (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well-known to those skilled in the art. Examples of suitable packaging virus lines include ψCrip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see, for example, Eglitis, et al. (1985) Science 230:1395-1398; Danos and Mulligan (1988) Proc. Natl. Acad. Sci. USA 85:6460-6464; Wilson, et al. (1988) Proc. Natl. Acad. Sci. USA 85:3014-3018; Armentano, et al. (1990) Proc. Natl. Acad. Sci. USA 87:6141-6145; Huber, et al. (1991) Proc. Natl. Acad. Sci. USA 88:8039-8043; Ferry, et al. (1991) Proc. Natl. Acad. Sci. USA 88:8377-8381; Chowdhury, et al. (1991) Science 254:1802-1805; van Beusechem, et al. (1992) Proc. Natl. Acad. Sci. USA 89:7640-7644; Kay, et al. (1992) Human Gene Therapy 3:641-647; Dai, et al. (1992) Proc. Natl. Acad. Sci. USA 89:10892-10895; Hwu, et al. (1993) J. Immunol. 150:4104-4115; U.S. Pat. No. 4,868,116; U.S. Pat. No. 4,980,286; WO 89/07136; WO 89/02468; WO 89/05345; and WO 92/07573).

Adeno-associated virus (AAV) can be used as a gene therapy vector for delivery of DNA for gene therapy purposes. AAV is a naturally occurring defective virus that requires another virus, such as an adenovirus or a herpes virus, as a helper virus for efficient replication and a productive life cycle (Muzyczka, et al. (1992) Curr. Topics Micro. Immunol. (1992) 158:97-129). AAV can be used to integrate DNA into non-dividing cells (see, for example, Flotte, et al. (1992) Am. J. Respir. Cell. Mol. Biol. 7:349-356; Samulski, et al. (1989) J. Virol. 63:3822-3828; and McLaughlin, et al. (1989) J. Virol. 62:1963-1973). An AAV vector such as that described in Tratschin, et al. (1985) Mol. Cell. Biol. 5:3251-3260 can be used to introduce DNA into cells (see, for example, Hermonat, et al. (1984) Proc. Natl. Acad. Sci. USA 81:6466-6470; Tratschin, et al. (1985) Mol. Cell. Biol. 4:2072-2081; Wondisford, et al. (1988) Mol. Endocrinol. 2:32-39; Tratschin, et al. (1984) J. Virol. 51:611-619; and Flotte, et al. (1993) J. Biol. Chem. 268:3781-3790). Lentiviral gene therapy vectors can also be adapted for use in the invention.

General methods for gene therapy are known in the art. See, for example, U.S. Pat. No. 5,399,346. A biocompatible capsule for delivering genetic material is described in WO 95/05452. Methods of gene transfer into hematopoietic cells have also previously been reported (see Clapp, et al. (1991) Blood 78:1132-1139; Anderson (2000) Science 288:627-9; Cavazzana-Calvo, et al. (2000) Science 288:669-72).

In assay methods of the invention, it is determined whether any agent so identified can be used for the prevention or treatment of angiogenesis-related disease, such as examining their effect(s) on disease symptoms in suitable angiogenesis-related disease animal model systems and their effect on VEGF production. The assay methods provided herein may similarly be used to identify and characterize compounds for the modulation of angiogenesis in a system, as well as for the modulation of VEGF production.

Agents which modulate the activity or expression of Shc are useful in preventing or treating diseases or process that are mediated by, or involve, angiogenesis. The present invention provides a method for preventing or treating an angiogenesis-mediated disease or process with an effective amount of an agent which modulates the expression or activity of Shc. Angiogenesis-mediated diseases or processs for which anti-angiogenic agents (i.e., Shc inhibitors) would be useful in alleviating the signs or symptoms of include, but are not limited to, tumor growth and proliferation (malignant or benign), blood bourne tumors such as leukemias, tumor metastasis, ocular angiogenic diseases, corneal graft rejection, retinal neovascularization due to macular degeneration, diabetic retinopathy, angiogenesis in the eye associated with infection, neovascular glaucoma, retrolental fibroplasia, rubeosis, angiogenic aspects of skin diseases, psoriasis, hemangiomas, acoustic neuromas, neurofibromas, rheumatoid arthritis, myocardial angiogenesis, intimal hyperplasia causing restenosis, endometriosis, pyogenic granuloma, scleroderma, trachoma, Osler-Weber Syndrome, atherosclerotic plaque neovascularization, telangiectasia, myocardial angiogenesis, hemophiliac joints, angiofibroma, wound granulation, intestinal adhesions, Crohn's disease, hypertrophic scars, keloids, cat scratch disease (Rochele minalia quintosa), ulcers (Helobacter pylori) and obesity (based on regulation of adipose tissue mass via vasculature (Rupnick, et al. (2002) Proc. Natl. Acad. Sci. USA 99:10730-10735).

Further, pro-angiogenic agents (i.e., Shc activators) would be useful for treating an angiogenesis-mediated disease or process such as stimulating wound healing, replacing clogged arteries to improve circulation in patients with arterial clogging, and treating various types of heart disease to promote the growth of blood vessels thereby reducing the need for bypass surgery.

In various embodiments, modulators of Shc activity, e.g., Shc inhibitors or activators), can be used therapeutically in formulations or medicaments to prevent or treat an angiogenesis-related disease or process. The invention provides corresponding methods of medical treatment, in which a therapeutic dose of a Shc inhibitor or activator is administered in a pharmacologically acceptable formulation, e.g., to a patient or subject in need thereof. Accordingly, the invention also provides therapeutic compositions containing an agent capable of modulating Shc expression or activity, e.g., a Shc inhibitor or activator, and a pharmacologically acceptable excipient or carrier. In one embodiment, such compositions include an Shc inhibitor or activator in a therapeutically or prophylactically effective amount sufficient to treat an angiogenesis-related disease or process. The therapeutic composition can be soluble in an aqueous solution at a physiologically acceptable pH.

An effective amount refers to an amount effective, at dosages and for periods of time necessary, to achieve the desired therapeutic result, such as a reduction or stimulation of angiogenesis and in turn a reduction in angiogenesis-related disease progression or stimulation of an angiogenesis-related process. A therapeutically effective amount of Shc inhibitor may vary according to factors such as the disease state, age, sex, and weight of the individual, and the ability of the agent to elicit a desired response in the individual. Dosage regimens can be adjusted to provide the optimum therapeutic response. A therapeutically effective amount is also one in which any toxic or detrimental effects of the agent are outweighed by the therapeutically beneficial effects. A prophylactically effective amount refers to an amount effective, at dosages and for periods of time necessary, to achieve the desired prophylactic result, such as preventing or inhibiting the rate of angiogenesis or angiogenesis-related disease onset or progression. A prophylactically effective amount can be determined as described above for the therapeutically effective amount. For any particular subject, specific dosage regimens can be adjusted over time according to the individual need and the professional judgement of the person administering or supervising the administration of the compositions.

Agents of the present invention can optionally be administered in conjunction with other therapeutic agents useful in the treatment of an angiogenesis-related disease or process.

The additional therapeutic agents can optionally be administered concurrently with the agents of the invention. As used herein, the word concurrently means sufficiently close in time to produce a combined effect (that is, concurrently can be simultaneously, or it can be two or more events occurring within a short time period before or after each other).

As used herein, pharmaceutically acceptable carrier or excipient includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well-known in the art. Except insofar as any conventional media or agent is incompatible with the active compound, use thereof in the pharmaceutical compositions of the invention is contemplated. Supplementary active compounds can also be incorporated into the compositions.

A further form of administration is to the eye. An Shc inhibiting agent can be delivered in a pharmaceutically acceptable ophthalmic vehicle, such that the agent is maintained in contact with the ocular surface for a sufficient time period to allow the agent to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically-acceptable ophthalmic vehicle may, for example, be an ointment, vegetable oil or an encapsulating material. Alternatively, the agent may be injected directly into the vitreous and aqueous humour. In a further alternative, the agent may be administered systemically, such as by intravenous infusion or injection, for treatment of the eye. In some embodiments, anti-angiogenic treatment with a Shc inhibiting agent can be undertaken following photodynamic therapy (such as is described in U.S. Pat. No. 5,798,349).

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, an Shc inhibitor or activator can be administered in a time-release formulation, for example in a composition which includes a slow release polymer. The active compounds can be prepared with carriers that will protect the agent against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art. A generally recognized compendium of such methods and ingredients is Remington: The Science and Practice of Pharmacy, Alfonso R. Gennaro, editor, 20th ed. Lippingcott Williams & Wilkins: Philadelphia, Pa., 2000.

Sterile injectable solutions can be prepared by incorporating the active compound (e.g. Shc inhibitor or activator) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation are vacuum drying and freeze-drying which yields a powder of the active ingredient plus any additional desired ingredient from a previously sterile-filtered solution thereof. In accordance with an alternative aspect of the invention, a Shc inhibitor or activator can be formulated with one or more additional compounds that enhance the solubility of the Shc inhibitor or activator.

In accordance with another aspect of the invention, therapeutic compositions of the present invention, containing a Shc inhibitor or activator, can be provided in containers or commercial packages which further contain instructions for use of the Shc inhibitor or activator for the inhibition or stimulation of angiogenesis, VEGF production or prevention or treatment of angiogenesis-related disease or process.

Accordingly, the invention further provides a commercial package containing an Shc inhibitor or activator or the above-mentioned composition together with instructions for the prevention or treatment of angiogenesis-related disease or process.

The invention is described in greater detail by the following non-limiting examples.

### EXAMPLE 1

### Materials and Methods

Antibodies. Antibody 144 was raised against a peptide in the carboxy-terminus of the Met protein (Rodrigues, et al. (1991) Mol. Cell. Biol. 11:2962-2970). Antibodies for phosphotyrosine and Grb2 were purchased from Transduction Labs (Lexington, Ky.), the VEGF antibody from Santa Cruz Biotechnology (Santa Cruz, Calif.) and the Neu antibody from Oncogene Science (Cambridge, Mass.). Rabbit polyclonal antibodies were raised to amino acid residues 366-473 of the human SH2 domain of Shc (Pelicci (1992) Cell 70:93-104).

DNA Constructs and Cell Lines. The cloning and characterization of the Tpr-Met, CSF-Met, and of the signal-specific binding variants are known in the art (Saucieri et al. (2002) supra; Fixman, et al. (1996) supra; Zhu, et al. (1994) J. Biol. Chem. 269:29943-29948). For the analysis conducted herein, the RTK oncoprotein Tpr-Met was primarily used. Tpr-Met, a transforming counterpart of the c-Met proto-oncogene detected in experimental and human cancer, is the result of a fusion of the Met kinase domain with a dimerization motif encoded by Tpr. In this rearrangement the exons encoding the Met extracellular, transmembrane and juxtamembrane domains are lost.

The cloning of the Grb2 and Shc RTK binding variants were performed essentially as described for the Tpr-Met variants, but using the Tyr^{1349/1356} CSF-Met receptor mutant as a recipient. All cells were cultured at 37° C. in Dulbecco's Modified Eagle's Medium supplemented with 10% fetal bovine serum. Expression of Tpr-Met in wild-type mouse embryo fibroblasts (MEF) or ShcA-deficient MEF cells was obtained by co-transfection of Tpr-Met cDNA with pLXSH vector using GENEPORTER (Gene Therapy System, San Diego, Calif.). Colonies resistant to hygromycin (150 μg/mL) were picked and expanded into cell lines. Generation and characterization of the activated wild-type Neu/ErbB-2 (NT), Neu tyrosine phosphorylation deficient (NYPD) and add-back mutants (NT-B Grb2 and NT-D Shc) are described in the art (Dankort (1997) Mol. Cell. Biol. 17:5410-5425; Dankort (2001) J. Biol. Chem. 276:38921-38928; Dankort (2001) Mol. Cell. Biol. 21:1540-1551). Expression of NT, NYPD and add-back mutants in Rat-1 fibroblast cells was obtained by co-transfection of corresponding cDNA with pLXSH vector by calcium phosphate method. Colonies resistant to hygromycin (150 μg/mL) were picked and expanded into cell lines. Transfection of 293T cells was performed by calcium phosphate method.

Tumorigenesis Assay. Fibroblast cells (10⁵ cells/100 μL) were injected subcutaneously into 3 to 4-week-old female nude mice (CD1 nu/nu; Charles River Laboratories, Wilmington, Mass.). The resulting tumors were measured periodically and allowed to grow until the tumors reached ˜1 cm³ or prior to ulceration, at which time the mice were sacrificed and the tumors collected for histochemical analysis. Tumor specimens were fixed overnight in 3.7% formaldehyde at 4° C., embedded in paraffin, and sectioned for hematoxylin and eosin (H&E) staining using standard histological procedures. Mean tumor volume was obtained from two independent experiments in which at least three mice were injected for each cell line

In vivo Angiogenesis Assay. Fibroblast cells (10⁵) mixed with 250 μL of serum-depleted MATRIGEL (Becton Dickinson Labware, Bedford, Mass.) were injected subcutaneously into 4 to 5-week-old CD1 nu/nu mice (Charles River), and animals were sacrificed after 10 days. The resulting MATRIGEL plugs were photographed and collected for histochemical analysis as described herein. For each cell line, at least six MATRIGEL plugs were analyzed.

Cell Lysate, Immunoprecipitation, In vitro Association Assay, and Immunoblotting. Preparation of cell lysates, immunoprecipitations, in vitro association assays and immunoblots were performed in accordance to methods well-established in the art (Fixman, et al. (1996) supra). Proteins were visualized using enhanced chemiluminescence (ECL, Amersham, Piscataway, N.J.) and films were digitized by scanning using ADOBE Photoshop. Each experiment was performed at least three times with independent preparation of cell lysates.

VEGF Protein Detection in Cell-Conditioned Media. Cells seeded at a density of 1 to 2×10⁶ cells/100-mm culture dish were the following day incubated for 48 hours in 4 mL of medium free of phenol red and serum. For stimulations, the ligand CSF (100 ng/mL) was added to the incubating medium. For the detection of VEGF protein, 1 to 1.4 mL of cleared, conditioned media was incubated for 1 hour at 4° C. with 25 μL of 50% heparin-SEPHAROSE. The heparin-SEPHAROSE protein complex was rinsed three times with washing buffer (20 mM Tris pH 7.5, 200 mM NaCl, 1 mM DTT and proteinase inhibitors) and bound proteins were eluted by addition of Laemmli sample buffer for immunoblot analysis with VEGF antibody.

Northern Blot Analysis. Total RNA was isolated from serum-starved (24-hour) cells expressing variant or control proteins by the TRIZOL method. RNAs (40 μg) were resolved by electrophoresis in formaldehyde containing agarose gels and transferred to a N+-HYBOND filter (Amersham). The blots were hybridized with a ³²P-labelled cDNA probe corresponding to the full-length VEGF transcript (Shweiki, et al. (1992) Nature 359:843-845).

### EXAMPLE 2

### Analysis of Fibroblasts Expressing Shc Binding Variants on Tumor Formation in Nude Mice

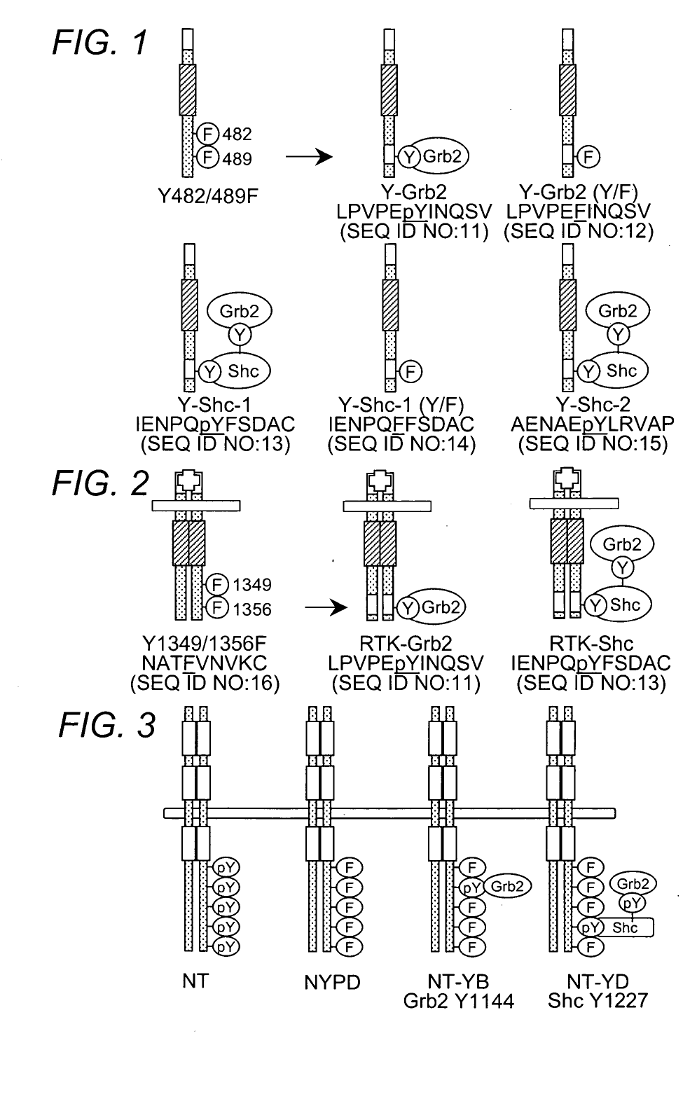

Using RTK oncoproteins specific for the binding of Grb2 or Shc (FIG. 1), it has been shown that the recruitment of Grb2 or Shc is sufficient to induce cell transformation, anchorage-independent growth, and experimental metastasis in vivo (Saucier, et al. (2002) supra). However, the individual contribution of Grb2 or Shc signals in tumorigenesis was unknown. To discriminate this, the tumorigenecity of fibroblast cell lines (10⁵ cells) expressing the Grb2 (Y-Grb2) or Shc (Y-Shc-1 or Y-Shc-2) binding variants of Trp-Met was evaluated following their subcutaneous injection into the flank of nude mice. The expression and phosphorylation level of wild-type Tpr-Met, Grb2 and Shc binding variants, and control proteins (Y-Grb2 Y/F, Y-Shc-1 Y/F, and Y⁴⁸²⁻⁴⁸⁹F) in fibroblast cell lines was determined. Lysates (500 μg) of fibroblast cell lines expressing each Tpr-Met binding variant or control protein were subjected to immunoprecipitation with an antibody specific for Met (Ab 144) and subsequently immunoblotted with the same antibody or anti-pTyr. It was determined that wild-type Tpr-Met, Grb2 and Shc binding variants, and control proteins were expressed and phosphorylated in each cell line generated.

Tumor growth (mm³) was measured after subcutaneous injection. It was observed that animals injected with cells expressing the non-transforming Tpr-Met cassette mutant (Tyr^{482/489}Phe), or corresponding negative controls for the Grb2 or Shc binding variants (Y-Grb2 Y/F or Y-Shc-1 Y/F), failed to develop tumors by 90 days post inoculation (Table 1). In contrast, fibroblasts transformed by the Grb2 or Shc binding variants grew as tumors, but with distinct latencies. Cells expressing Shc binding variants (Y-Shc-1 or Y-Shc-2) induced palpable tumors with short latency (˜7 days), whereas the appearance of tumors with cells expressing the Grb2 binding variant was delayed to ˜24 days after subcutaneous injection (Table 1).

TABLE 1

| Cell Line | # Tumor/# injection | Tumor Latency (Days) |
|---|---|---|
| Y-Grb2 | 6/6 | 24 ± 3.1 |
| Y-Grb2 (Y/F) | 0/6 | >90 |
| Y-Shc-1 | 6/6 | 6.0 ± 1.1 |
| Y-shc-1 (Y/F) | 0/6 | >90 |
| Y-Shc-2 | 6/6 | 7.3 ± 1.2 |
| Y482/489F | 0/6 | >90 |

The tumorigenecity of Fr3T3 cells expressing signal protein binding RTK oncoprotein was evaluated following their injection subcutaneously into nude mice. These results demonstrate that fibroblasts expressing Shc binding variants form tumors in nude mice with short latency.

### EXAMPLE 3

### Effect of Fibroblasts Expressing Shc Binding Variants on Induction of an Angiogenic Response in Nude Mice and on VEGF Production

Since the transforming activity of the Y-Grb2 and Y-Shc-2 variants in in vitro culture assays are similar (Saucier, et al. (2002) supra), the difference observed in the latency of tumor formation in vivo is unexpected. As described herein, an in vivo angiogenesis MATRIGEL plug assay was performed using standard methods (Passaniti, et al. (1992) Lab. Invest. 67:519-528). Fibroblasts expressing the Grb2 or Shc binding variants, or control proteins, were mixed with a MATRIGEL solution depleted of growth factor, allowing the maintenance of cells within the MATRIGEL after subcutaneous injection into nude mice. The presence of blood vessels within MATRIGEL plugs was analyzed 10 days after subcutaneous injection. Upon gross and histological examination it was observed that MATRIGEL plugs of cells expressing the Shc binding variants (Y-Shc-1 or Y-Shc-2) were red and contained many blood vessels. In contrast, MATRIGEL plugs containing fibroblasts transformed by the Grb2 binding variant, or controls, remained clear and were poorly vascularized.

Subsequently, the ability of cell lines expressing the Grb2, or Shc binding variants, to produce VEGF protein in their conditioned media was examined. VEGF protein (VEGF165, ˜23 kDa) was readily detected in the conditioned media of cells expressing Shc binding variants (Y-Shc-1 and Y-Shc-2), whereas the level of VEGF protein produced by fibroblasts expressing the Grb2 binding variant or controls was barely detectable or absent. An increase in VEGF protein is often associated with an increase in VEGF mRNA (Ferrara (1999) J. Mol. Med. 77:527-543; Toyoda, et al. (2001) FEBS Lett. 509:95-100; Dong, et al. (2001) Cancer Res. 61:5911-5918; Calza, et al. (2001) Proc. Natl. Acad. Sci. USA 98:4160-4165; Maity, et al. (2000) Cancer Res. 60:5879-5886; Wang, et al. (1999) Cancer Res. 59:1464-1472; Seghezzi, et al. (1998) J. Cell Biol. 141:1659-1673; Miele, et al. (2000) J. Biol. Chem. 275:21695-21702). Consistent with this, the level of VEGF mRNA detected in cells expressing the Shc binding variants (Y-Shc-1 or Y-Shc-2) was significantly enhanced when compared to fibroblasts expressing the transforming Grb2 binding variant or controls. Hence, the angiogenenic properties of cells expressing the Shc binding variants reflect their ability to produce VEGF protein and this correlates with the rapid growth of these cells as tumors in vivo (Table 1).

### EXAMPLE 4

### Effect of Shc and Grb2 Binding to a Ligand-Activated RTK on Induction of VEGF Production

The Grb2 and Shc binding variants are constitutively activated RTK oncoproteins derived from the Met/HGF receptor oncoprotein, Tpr-Met (Saucier, et al. (2002) supra). This RTK oncoprotein lacks the transmembrane domain of the Met receptor and is a cytosolic protein (Saucier, et al. (2002) supra). To determine whether the recruitment of Shc or Grb2 to a transmembrane RTK, activated in a ligand-dependent manner, was sufficient to induce VEGF production, a CSF-Met receptor chimera mutant lacking the two critical tyrosines (Met-Tyr^{1349/1356}Phe, (Zhu, et al. (1994) J. Biol. Chem. 269:29943-29948)) was engineered to specifically bind either, Grb2 or Shc (RTK-Grb2 or RTK-Shc, respectively; FIG. 2). The binding specificity of the Grb2 or Shc binding RTK variants was confirmed by in vitro association and coimmunoprecipitation assays following transient transfection in 293T cells. Lysates prepared from 293T cells transiently transfected with the RTK-Grb2, RTK-Shc or Met-Y1349/1356F mutant were subjected to immunoprecipitation with an antibody specific for Met, and immunoblotted with anti-Met or anti-pTyr. The level of Grb2 or Shc proteins associated with the RTK-Grb2 or RTK-Shc RTKs, or Met-Y1349/1356F mutant was detected in lysates of 293T cells subjected to an immunoprecipitation with an antibody specific for Met followed by immunoblotted analysis with a Grb2 or Shc specific antibodies. The expression level of Grb2 and Shc in the cells was detected by immunoblot analyses conducted with a Grb2 or Shc specific antibody.

The ability of the Grb2 or Shc binding RTK variants to induce the expression of VEGF protein upon ligand stimulation was tested in Rat-1 fibroblasts. VEGF protein, detected by immunoblot analysis using an antibody specific for the VEGF protein after enrichment with heparin precipitation, was found in the conditioned media of two independent cell populations expressing the RTK-Shc but not in cells expressing the RTK-Grb2 or the Met-Tyr^{1349/1356}Phe mutant after 48 hours of stimulation. The induction of VEGF production by cells expressing the RTK-Shc binding variant was not observed in non-stimulated cells. These results further demonstrate that the recruitment of Shc but not of Grb2, to a transmembrane spanning RTK, is sufficient to enhance the production of the VEGF protein upon ligand activation.

### EXAMPLE 5

### Effect of Shc and Grb2 Binding to the Neu/erbB2 RTK on Induction of VEGF Production

The Neu/c-ErbB-2/HER2 is a member of the epidermal growth factor receptor (EGFR) family, which are transmembrane RTKs (Olayioye (2000) EMBO J. 19:3159-3167). Amplification of Neu/c-ErbB-2/HER2 RTK is implicated in the etiology of ovarian and breast cancers and correlates with a poor clinical prognosis in breast cancer patients (Slamon (1987) Science 235:177-182; Slamon (1989) Science 244:707-712). Direct evidence for a role for Shc- and Grb2-dependent signals in Neu/erbB2/HER2-mediated mammary tumorigenesis has been established with transgenic mice which develop mammary tumors when expressing in their mammary epithelia an activated Neu/ErbB-2 RTK add-back mutant in which only the Shc (YD strain) or Grb2 (YB strain) binding site was reintroduced (Dankort (2001) Mol. Cell Biol. 21:1540-1551). Importantly, a shorter latency of mammary tumor development and enhanced tumor burden was observed in transgenic mice expressing an activated Neu/ErbB2 RTK mutant in which only the Shc binding site was reintroduced (YD), when compared to a mutant of Neu/ErbB2 that binds Grb2 (YB).

Thus, the level of VEGF produced after 48 hours in the conditioned media was compared amongst Rat-1 fibroblast cells expressing activated wild-type Neu/Erb2 (NT), NT-YB (Grb2) or NT-YD (Shc) add-back mutants, or of the NYPD mutant (FIG. 3). It was observed that the level of VEGF protein was increased downstream of the NT-YD mutant in which only the Shc binding site was reintroduced. In contrast, comparable to cells expressing the signaling-deficient NT-NYPD mutant, no detectable VEGF protein was produced in the conditioned media of cells expressing a Neu/ErbB2/HER2 add-back RTK mutant binding to Grb2. This demonstrates, in another RTK context, the importance of Shc but not Grb2-dependent signals for the induction of VEGF production.

### EXAMPLE 6

### Analysis of Met Receptor-Mediated VEGF Production With Respect to Angiogenesis and Grb2.

Results pertaining to the signaling-specific RTKs indicate that the recruitment of Shc, but not of Grb2 to RTKs plays a critical role in the induction of VEGF protein, and tumor angiogenesis. In support of this, it has been shown that fibroblasts expressing a mutant Tpr-Met oncoprotein that fails to bind to Grb2 (Tpr-Met ΔGrb2 (Fixman, et al. (1996) supra, Ponzetto, et al. (1996) supra)), produced similar levels of VEGF than cells expressing wild-type Tpr-Met, which recruits both Grb2 and Shc. In contrast, cells expressing a mutant Tpr-Met (Tyr^{482/489}Phe) that is unable to recruit Grb2 and Shc (Saucier, et al. (2002) supra; Fixman, et al. (1996) supra, Ponzetto, et al. (1996) supra), failed to produce VEGF. Consistent with this, MATRIGEL plugs containing cells expressing the wild-type Tpr-Met or the Tpr-Met ΔGrb2 mutant were red and abundantly infiltrated by blood vessels, whereas MATRIGEL plugs of cells expressing the Tyr^{482/482}Phe Tpr-Met mutant were translucent and poorly vascularized. Hence, the induction of VEGF production and the consequent angiogenic activity of the Tpr-Met oncoprotein are independent of its coupling to the Grb2 adaptor protein.

### EXAMPLE 7

### Analysis of Met Receptor-Mediated VEGF Production and Shc Signaling

To define the requirement for Shc in Met-induced VEGF production, the Tpr-Met oncogene was stably expressed in mouse embryonic fibroblasts (MEF) derived from wild-type (+/+), or ShcA-deficient mouse embryos (−/−), as well as in ShcA-deficient MEFs expressing the p52/p46 ShcA isoforms (−/− p52Shc)(Lai & Pawson (2000) supra). When Shc was expressed, its level of phosphorylation on tyrosine residues was elevated by the expression of Tpr-Met. An increase in the production of VEGF was induced by the expression of Tpr-Met in wild-type MEFs, but not in the ShcA-deficient cells. Notably, in ShcA-deficient MEFs transfected with the p52ShcA gene, the induction of VEGF production by the Tpr-Met oncoprotein was rescued. These results identify Shc as an intermediate required for VEGF production downstream from Tpr-Met.

### EXAMPLE 8

### Analysis of the Requirement of Shc for Induction of VEGF by Serum Growth Factors

To define the requirement of Shc for serum-induced VEGF production, MEFs derived from wild-type (+/+), ShcA-deficient mouse embryos (−/−), or ShcA-deficient MEFs expressing the p52 ShcA gene (−/− p52Shc) were cultivated in the presence or absence of 10% fetal bovine serum. An increase in the production of VEGF was induced in the presence of serum in wild-type MEFs, but not in the ShcA-deficient cells. Notably, the induction of VEGF production by serum was rescued in ShcA-deficient MEFs when p52ShcA gene was expressed. Thus, these results demonstrate that Shc is required for induction of VEGF by serum growth factors.

### EXAMPLE 9

### Analysis of the TSP-1 Expression

It was determined whether the recruitment of Shc or Grb2 to a RTK oncoprotein was sufficient to mediate a downregulation of the angiogenic inhibitor TSP-1, the level of TSP-1 present in lysate of serum-starved Fr3T3 fibroblasts expressing the Grb2- or Shc-binding variants was examined by immunoblot analysis using a TSP-1 specific antibody (Lab Vision Corp., CA). TSP-1 protein was readily detected in lysate of the Grb2-binding variant or control (Fr3T3 or Tpr-Met Y482/489F). In contrast, the level of TSP-1 protein was drastically reduced in cells expressing wild-type Tpr-Met or the two Shc-specific binding variants (Y-Shc-1 or Y-Shc-2). These results demonstrate that the activation of Shc signaling pathways is sufficient to induce downregulation of TSP-1.

It was subsequently determined whether downregulation of TSP-1, as mediated by the Met receptor, was independent of its coupling to Grb2. When expressed in fibroblasts, it was found that a wild-type Met RTK oncoprotein or a mutant form deficient at binding Grb2 (ΔGrb2, N491H), but which retained Shc binding, mediated downregulation of TSP-1 when compared to the parental cells (Fr3T3). Conversely, the level of TSP-1 protein was not downregulated by the expression of a Met RTK oncoprotein mutant unable to recruit Grb2 and Shc (Tyr^{482/489}Phe). Thus, these data demonstrate that the TSP-1 downregulation, mediated by the Met receptor, is independent of its coupling to Grb2.

To define the requirement for Shc in growth factor signal-induced downregulation of TSP-1, the level of TSP-1 protein produced in MEF derived from ShcA-deficient mouse embryos (−/−), as well as in ShcA-deficient MEFs expressing the p52/p46 ShcA isoforms (−/− p52Shc) grown in the presence or absence of 10% fetal bovine serum was examined. It was observed that serum failed to downregulate TSP-1 expression in the ShcA-deficient cells. However, expression of p52ShcA in ShcA-deficient MEFs enhanced TSP-1 expression, which was abrogated in presence of serum. Thus, these results demonstrate that downregulation of TSP-1 induced by serum-derived growth factors is dependent on Shc signaling pathways.

### EXAMPLE 10

### Effect of Shc Signaling Pathways on Regulation of Fibroblast Growth Factor-2 and Angiopoietin Proteins

The level of gene expression of various regulators of angiogenesis was determined. Phosphorylation and expression levels of wild-type Tpr-Met, Grb2 and Shc binding variants (Y-Grb2, Y-Shc-1, and Y-Shc-2) and negative controls for the Grb2 or Shc binding variants (Y-Grb2 Y/F and Y-Shc-1 Y/F) were examined in NIH-3T3 fibroblast stable cell populations. It was determined that wild-type Tpr-Met, Grb2 and Shc binding variants, and control proteins were expressed and phosphorylated in each cell population generated.

Subsequently, the level of VEGF protein produced in the conditioned media of NIH-3T3 fibroblast cells expressing the above-referenced wild-type Tpr-Met, Tpr-Met variant forms, and control proteins was determined. Increases in the production of VEGF protein were observed in cells expressing variants Y-Shc-1 and Y-Shc-2 as compared to wild-type Tpr-Met and VEGF protein levels were reduced in cells expressing Y-Grb2 and negative control proteins. Further, the relative level of VEGF mRNA, normalized to GAPDH, was detected by real-time RT-PCR using total RNA prepared from cells expressing wild-type Tpr-Met or variants. There was at least a five-fold induction of VEGF mRNA in cells expressing Y-Shc-1, Y-Shc-2 or wild-type Tpr-Met as compared to control cells (Y-Shc-1 Y/F, pLXSN or parental cells). There was approximately a two-fold increase in VEGF mRNA levels in cells expressing the Grb2 binding RTK oncoprotein (Y-Grb2).

The level of TSP-1, Ang-1, Ang-2 and FGF-2 mRNA was detected by RT-PCR analysis of RNA samples of cells expressing wild-type Tpr-Met or variant forms. The level of GAPDH was detected as control for loading. It was determined that activation of Shc signaling pathways by RTK oncoproteins (i.e., in cells expressing Y-Shc-1 and Y-Shc-2 or wild-type Tpr-Met) induces upregulation FGF-2 and Angiopoietin-2, as well as downregulation of TSP-1 and angiopoietin-1.

### EXAMPLE 11

### RNAi-Based Inhibition of Shc

To ablate Shc in cell lines or animal tumor models known to induce an angiogenic response, an RNA interfering strategy is used. Stable repression of Shc expression is obtained by delivery in cells of siRNA using a DNA vector-based method where the U6 RNA promoter drives the expression of RNAs predicted to form small hairpins containing 27 to 29-nt stems matching a coding region of the Shc gene (Paddison, et al. (2002) Gene Dev. 16:948-958). RNAi oligo Retriever program (www.cshl.org/public/SCIENCE/hannon.html) may be used to design small hairpin PCR DNA primers derived from the coding sequence of Shc. Each hairpin primer contains a 27 to 29-nt inverted repeats complementary to the Shc coding sequence separated by an 8-nt spacer loop region (containing a HindIII site to screen for the presence of hairpins), a transcriptional termination signal at the 3′-end of the inverted repeat, and a 21-nt region complementary to the human U6 snRNA promoter sequence compatible for PCR cloning of the shRNA sequences downstream of the human U6 promoter. The cloning of the human RNA U6 promoter in front of the Shc-specific small hairpin DNA sequence is carried out by PCR using the pGEM1 plasmid containing the human U6 locus as template, and a SP6 primer complementary to the upstream portion of the U6 promoter compatible for pENTR/D TOPO-cloning (Gateway system, INVITROGEN). The resulting ˜600-bp PCR U6-shRNA cassette is cloned into pENTR/TOPO-D vector (INVITROGEN) using the directional TOPO-cloning method according to manufacturer's instructions. This construct is then used to transport any of the U6-shRNA cassette to suitable recipient retroviral vectors of choice containing a gateway destination cassette (e.g., pLXSN, pLXSH, pBabePuro, pMSCV).

The efficiency of the different shRNA molecules to reduce endogenous or overexpressed Shc is determined at the mRNA (by RT-PCR or northern blot analysis) or protein levels (by immunoblot analysis). For the shRNA molecules capable of depleting cellular Shc, their abilities to reduce VEGF production and angiogenesis are tested using fibroblast cell models, where the production of VEGF protein and their in vivo angiogenic responses is dependent on Shc (e.g., fibroblasts expressing Tpr-Met, variants Y-Shc-1 or Y-Shc-2, or Neu add-back mutants). Furthermore, these shRNA molecules are further tested in well-established and characterized human tumor cell lines (from NCI-60 collection or a panel of breast cancer cell lines) determined to produce VEGF protein and to induce an angiogenic responses in vivo. For each cell line tested, the levels of VEGF produced and the ability of these cells to induce angiogenic responses in the in vivo angiogenesis MATRIGEL plug assays are determined before and after depletion of cellular Shc.

What is claimed is:

1. A method for modulating angiogenesis in a cell, tissue, or subject comprising contacting a cell, tissue, or subject with an agent which regulates the expression or activity of Shc thereby altering the production of VEGF or the expression of a modulator of angiogenesis so that angiogenesis in said cell, tissue, or subject is modulated.

2. The method of claim 1, wherein the modulator of angiogenesis is fibroblast growth factor-2, angiopoietin-2, thrombospondin-1 or angiopoietin-1.

3. A method for preventing or treating an angiogenesis-related disease or process in a subject comprising administering to a subject an agent which modulates the expression or activity of Shc thereby altering the production of VEGF or expression of a modulator of angiogenesis so that the angiogenesis-related disease or process in said subject is prevented or treated.

4. The method of claim 3, wherein the modulator of angiogenesis is fibroblast growth factor-2, angiopoietin-2, thrombospondin-1 or angiopoietin-1.

5. A method for identifying an agent that modulates angiogenesis comprising contacting a first cell expressing Shc with a test agent and measuring the expression of a modulator of angiogenesis in said first cell as compared to a second cell expressing Shc not contacted with the test agent, wherein a lower or higher measured activity in the first cell, as compared to the measured activity in the second cell is indicative of an agent which modulates angiogenesis.

6. The method of claim 3, wherein the modulator of angiogenesis is VEGF, fibroblast growth factor-2, angiopoietin-2, thombrospondin-1 or angiopoietin-1.

7. An agent identified by the method of claim 6.


SEQUENCE LISTING (16 sequences)

SEQ ID NO: 1 - DNA, 3031 bp, Homo sapiens
gcggtaacctaagctggcagtggcgtgatccggcaccaaatcggcccgcggtgcgtgcggagactccatgaggccctggacatgaacaagctgagtggaggcggcgggcgcaggactcgggtggaagggggccagcttgggggcgaggagtggacccgccacgggagctttgtcaataagcccacgcggggctggctgcatcccaacgacaaagtcatgggacccggggtttcctacttggttcggtacatgggttgtgtggaggtcctccagtcaatgcgtgccctggacttcaacacccggactcaggtcaccagggaggccatcagtctggtgtgtgaggctgtgccgggtgctaagggggcgacaaggaggagaaagccctgtagccgcccgctcagctctatcctggggaggagtaacctgaaatttgctggaatgccaatcactctcaccgtctccaccagcagcctcaacctcatggccgcagactgcaaacagatcatcgccaaccaccacatgcaatctatctcatttgcatccggcggggatccggacacagccgagtatgtcgcctatgttgccaaagaccctgtgaatcagagagcctgccacattctggagtgtcccgaagggcttgcccaggatgtcatcagcaccattggccaggccttcgagttgcgcttcaaacaatacctcaggaacccacccaaactggtcacccctcatgacaggatggctggctttgatggctcagcatgggatgaggaggaggaagagccacctgaccatcagtactataatgacttcccggggaaggaaccccccttggggggggtggtagacatgaggcttcgggaaggagccgctccaggggctgctcgacccactgcacccaatgcccagacccccagccacttgggagctacattgcctgtaggacagcctgttgggggagatccagaagtccgcaaacagatgccacctccaccaccctgtccaggcagagagctttttgatgatccctcctatgtcaacgtccagaacctagacaaggcccggcaagcagtgggtggtgctgggccccccaatcctgctatcaatggcagtgcaccccgggacctgtttgacatgaagcccttcgaagatgctcttcgggtgcctccacctccccagtcggtgtccatggctgagcagctccgaggggagccctggttccatgggaagctgagccggcgggaggctgaggcactgctgcagctcaatggggacttcttggtacgggagagcacgaccacacctggccagtatgtgctcactggcttgcagagtgggcagcctaagcatttgctactggtggaccctgagggtgtggttcggactaaggatcaccgctttgaaagtgtcagtcaccttatcagctaccacatggacaatcacttgcccatcatctctgcgggcagcgaactgtgtctacagcaacctgtggagcggaaactgtgatctgccctagcgctctcttccagaagatgccctccaatcctttccaccctattccctaactctcgggacctcgtttgggagtgttctgtgggcttggccttgtgtcagagctgggagtagcatggactctgggtttcatatccagctgagtgagagggtttgagtcaaaagcctgggtgagaatcctgcctctccccaaacattaatcaccaaagtattaatgtacagagtggcccctcacctgggcctttcctgtgccaacctgatgccccttccccaagaaggtgagtgcttgtcatggaaaatgtcctgtggtgacaggcccagtggaacagtcacccttctgggcaagggggaacaaatcacacctctgggcttcagggtatcccagacccctctcaacacccgccccccccatgtttaaactttgtgcctttgaccatctcttaggtctaatgatattttatgcaaacagttcttggacccctgaattcttcaatgacagggatgccaacaccttcttggcttctgggacctgtgttcttgctgagcaccctctccggtttgggttgggataacagaggcaggagtggcagctgtcccctctccctggggatatgcaacccttagagattgccccagagccccactcccggccaggcgggagatggacccctcccttgctcagtgcctcctggccggggcccctcaccccaaggggtctgtatatacatttcataaggcctgccctcccatgttgcatgcctatgtactctgcgccaaagtgcagcccttcctcctgaagcctctgccctgcctccctttctgggagggcggggtgggggtgactgaatttgggcctcttgtacagttaactctcccaggtggattttgtggaggtgagaaaaggggcattgagactataaagcagtagacaatccccacataccatctgtagagttggaactgcattcttttaaagttttatatgcatatattttagggctgctagacttactttcctattttcttttccattgcttattcttgagcacaaaatgataatcaattattacatttatacatcacctttttgacttttccaagcccttttacagctcttggcattttcctcgcctaggcctgtgaggtaactgggatcgcaccttttataccagagacctgaggcagatgaaatttatttccatctaggactagaaaaacttgggtctcttaccgcgagactgagaggcagaagtcagcccgaatgcctgtcagtttcatggaggggaaacgcaaaacctgcagttcctgagtaccttctacaggcccggcccagcctaggcccggggtggccacaccacagcaagccggccccccctcttttggccttgtggataagggagagttgaccgttttcatcctggcctccttttgctgtttggatgtttccacgggtctcacttataccaaagggaaaactcttcattaaagtcccgtatttcttctaaaaaaaaaaaaaaaaaaa

SEQ ID NO: 2 - protein, 473 aa, Homo sapiens
MNKLSGGGGRRTRVEGGQLGGEEWTRHGSFVNKPTRGWLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRNPPKLVTPHDRMAGFDGSAWDEEEEEPPDHQYYNDFPGKEPPLGGVVDMRLREGAAPGAARPTAPNAQTPSHLGATLPVGQPVGGDPEVRKQMPPPPPCPGRELFDDPSYVNVQNLDKARQAVGGAGPPNPAINGSAPRDLFDMKPFEDALRVPPPPQSVSMAEQLRGEPWFHGKLSRREAEALLQLNGDFLVRESTTTPGQYVLTGLQSGQPKHLLLVDPEGVVRTKDHRFESVSHLISYHMDNHLPIISAGSELCLQQPVERKL

SEQ ID NO: 3 - DNA, 3664 bp, Homo sapiens
atggggcctgaaactgtctgggtctgagctggggagcggaagccacttgtccctctccctccccaggacttctgtgactcctgggccacagaggtccaaccagggtaagggcctggggataccccctgcctggcccccttgcccaaactggcaggggggccaggctgggcagcagcccctctttcacctcaactatggatctcctgccccccaagcccaagtacaatccactccggaatgagtctctgtcatcgctggaggaaggggcttctgggtccacccccccggaggagctgccttccccatcagcttcatccctggggcccatcctgcctcctctgcctggggacgatagtcccactaccctgtgctccttcttcccccggatgagcaacctgaggctggccaacccggctggggggcgcccagggtctaagggggagccaggaagggcagctgatgatggggaggggatcgatggggcagccatgccagagtcaggccccctacccctcctccaggacatgaacaagctgagtggaggcggcgggcgcaggactcgggtggaagggggccagcttgggggcgaggagtggacccgccacgggagctttgtcaataagcccacgcggggctggctgcatcccaacgacaaagtcatgggacccggggtttcctacttggttcggtacatgggttgtgtggaggtcctccagtcaatgcgtgccctggacttcaacacccggactcaggtcaccagggaggccatcagtctggtgtgtgaggctgtgccgggtgctaagggggcgacaaggaggagaaagccctgtagccgcccgctcagctctatcctggggaggagtaacctgaaatttgctggaatgccaatcactctcaccgtctccaccagcagcctcaacctcatggccgcagactgcaaacagatcatcgccaaccaccacatgcaatctatctcatttgcatccggcggggatccggacacagccgagtatgtcgcctatgttgccaaagaccctgtgaatcagagagcctgccacattctggagtgtcccgaagggcttgcccaggatgtcatcagcaccattggccaggccttcgagttgcgcttcaaacaatacctcaggaacccacccaaactggtcacccctcatgacaggatggctggctttgatggctcagcatgggatgaggaggaggaagagccacctgaccatcagtactataatgacttcccggggaaggaaccccccttggggggggtggtagacatgaggcttcgggaaggagccgctccaggggctgctcgacccactgcacccaatgcccagacccccagccacttgggagctacattgcctgtaggacagcctgttgggggagatccagaagtccgcaaacagatgccacctccaccaccctgtccaggcagagagctttttgatgatccctcctatgtcaacgtccagaacctagacaaggcccggcaagcagtgggtggtgctgggccccccaatcctgctatcaatggcagtgcaccccgggacctgtttgacatgaagcccttcgaagatgctcttcgggtgcctccacctccccagtcggtgtccatggctgagcagctccgaggggagccctggttccatgggaagctgagccggcgggaggctgaggcactgctgcagctcaatggggacttcttggtacgggagagcacgaccacacctggccagtatgtgctcactggcttgcagagtgggcagcctaagcatttgctactggtggaccctgagggtgtggttcggactaaggatcaccgctttgaaagtgtcagtcaccttatcagctaccacatggacaatcacttgcccatcatctctgcgggcagcgaactgtgtctacagcaacctgtggagcggaaactgtgatctgccctagcgctctcttccagaagatgccctccaatcctttccaccctattccctaactctcgggacctcgtttgggagtgttctgtgggcttggccttgtgtcagagctgggagtagcatggactctgggtttcatatccagctgagtgagagggtttgagtcaaaagcctgggtgagaatcctgcctctccccaaacattaatcaccaaagtattaatgtacagagtggcccctcacctgggcctttcctgtgccaacctgatgccccttccccaagaaggtgagtgcttgtcatggaaaatgtcctgtggtgacaggcccagtggaacagtcacccttctgggcaagggggaacaaatcacacctctgggcttcagggtatcccagacccctctcaacacccgccccccccatgtttaaactttgtgcctttgaccatctcttaggtctaatgatattttatgcaaacagttcttggacccctgaattcttcaatgacagggatgccaacaccttcttggcttctgggacctgtgttcttgctgagcaccctctccggtttgggttgggataacagaggcaggagtggcagctgtcccctctccctggggatatgcaacccttagagattgccccagagccccactcccggccaggcgggagatggacccctcccttgctcagtgcctcctggccggggcccctcaccccaaggggtctgtatatacatttcataaggcctgccctcccatgttgcatgcctatgtactctgcgccaaagtgcagcccttcctcctgaagcctctgccctgcctccctttctgggagggcggggtgggggtgactgaatttgggcctcttgtacagttaactctcccaggtggattttgtggaggtgagaaaaggggcattgagactataaagcagtagacaatccccacataccatctgtagagttggaactgcattcttttaaagttttatatgcatatattttagggctgctagacttactttcctattttcttttccattgcttattcttgagcacaaaatgataatcaattattacatttatacatcacctttttgacttttccaagcccttttacagctcttggcattttcctcgcctaggcctgtgaggtaactgggatcgcaccttttataccagagacctgaggcagatgaaatttatttccatctaggactagaaaaacttgggtctcttaccgcgagactgagaggcagaagtcagcccgaatgcctgtcagtttcatggaggggaaacgcaaaacctgcagttcctgagtaccttctacaggcccggcccagcctaggcccggggtggccacaccacagcaagccggccccccctcttttggccttgtggataagggagagttgaccgttttcatcctggcctccttttgctgtttggatgtttccacgggtctcacttataccaaagggaaaactcttcattaaagtccgtatttcttctaaaaaaaaaaaaaaaaaaatacatttatacatcacctttttgacttttccaagcccttttacagctcttggcattttcctcgcctaggcctgtgaggtaactgggatcgcaccttttataccagagacctgaggcagatgaaatttatttccatctaggactagaaaaacttgggtctcttaccgcgagactgagaggcagaagtcagcc

SEQ ID NO: 4 - protein, 583 aa, Homo sapiens
MDLLPPKPKYNPLRNESLSSLEEGASGSTPPEELPSPSASSLGPILPPLPGDDSPTTLCSFFPRMSNLRLANPAGGRPGSKGEPGRAADDGEGIDGAAMPESGPLPLLQDMNKLSGGGGRRTRVEGGQLGGEEWTRHGSFVNKPTRGWLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRNPPKLVTPHDRMAGFDGSAWDEEEEEPPDHQYYNDFPGKEPPLGGVVDMRLREGAAPGAARPTAPNAQTPSHLGATLPVGQPVGGDPEVRKQMPPPPPCPGRELFDDPSYVNVQNLDKARQAVGGAGPPNPAINGSAPRDLFDMKPFEDALRVPPPPQSVSMAEQLRGEPWFHGKLSRREAEALLQLNGDFLVRESTTTPGQYVLTGLQSGQPKHLLLVDPEGVVRTKDHRFESVSHLISYHMDNHLPIISAGSELCLQQPVERKL

SEQ ID NO: 5 - DNA, 1879 bp, Homo sapiens
agctatgaatctcctgccccccaagcccaagtacaatccactccggaatgagtctctgtcatcgatggaggaaggggcttctgggtccacccccccggaggagctgccttccccaccagcttcatccctggggcccatcctgcctcctctgcctggggacgatagtcccactaccctgtgctccttcttcccccggatgagcaacctgaggctggccaacccggctggggggcgcccagggtctaagggggagccaggaagggcagctgatgatggggaggggatcgtaggggcagccatgccagactcaggccccctacccctcctccaggacatgaacaagctgagtggaggcggcgggcgcaggactcgggtggaagggggccagcttgggggcgaggagtggacccgccacgggagctttgtcaataagcccacgcggggctggctgcatcccaacgacaaagtcatgggacccggggtttcctacttggttcggtacatgggttgtgtggaggtcctccagtcaatgcgtgccctggacttcaacacccggactcaggtcaccagggaggccatcagtctggtgtgtgaggctgtgccgggtgctaagggggcgacaaggaggagaaagccctgtagccgcccgctcagctctatcctggggaggagtaacctgaaatttgctggaatgccaatcactctcaccgtctccaccagcagcctcaacctcatggccgcagactgcaaacagatcatcgccaaccaccacatgcaatctatctcatttgcatccggcggggatccggacacagccgagtatgtcgcctatgttgccaaagaccctgtgaatcagagagcctgccacattctggagtgtcccgaagggcttgcccaggatgtcatcagcaccattggccaggccttcgagttgcgcttcaaacaatacctcaggaacccacccaaactggtcacccctcatgacaggatggctggctttgatggctcagcatgggatgaggaggaggaagagccacctgaccatcagtactataatgacttcccggggaaggaaccccccttggggggggtggtagacatgaggcttcgggaaggagccgctccaggggctgctcgacccactgcacccaatgcccagacccccagccacttgggagctacattgcctgtaggacagcctgttgggggagatccagaagtccgcaaacagatgccacctccaccaccctgtccaggcagagagctttttgatgatccctcctatgtcaacgtccagaacctagacaaggcccggcaagcagtgggtggtgctgggccccccaatcctgctatcaatggcagtgcaccccgggacctgtttgacatgaagcccttcgaagatgctcttcgggtgcctccacctccccagtcggtgtccatggctgagcagctccgaggggagccctggttccatgggaagctgagccggcgggaggctgaggcactgctgcagctcaatggggacttcttggtacgggagagcacgaccacacctggccagtatgtgctcactggcttgcagagtgggcagcctaagcatttgctactggtggaccctgagggtgtggttcggactaaggatcaccgctttgaaagtgtcagtcaccttatcagctaccacatggacaatcacttgcccatcatctctgcgggcagcgaactgtgtctacagcaacctgtggagcggaaactgtgatctgccctagcgctctcttccagaagatgccctccaatcctttccaccctattccctaactctcgggacctcgtttgggagtgttctgtgggcttggccttgtgtcagagctgggagtagcat

SEQ ID NO: 6 - protein, 583 aa, Homo sapiens
MNLLPPKPKYNPLRNESLSSMEEGASGSTPPEELPSPPASSLGPILPPLPGDDSPTTLCSFFPRMSNLRLANPAGGRPGSKGEPGRAADDGEGIVGAAMPDSGPLPLLQDMNKLSGGGGRRTRVEGGQLGGEEWTRHGSFVNKPTRGWLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRNPPKLVTPHDRMAGFDGSAWDEEEEEPPDHQYYNDFPGKEPPLGGVVDMRLREGAAPGAARPTAPNAQTPSHLGATLPVGQPVGGDPEVRKQMPPPPPCPGRELFDDPSYVNVQNLDKARQAVGGAGPPNPAINGSAPRDLFDMKPFEDALRVPPPPQSVSMAEQLRGEPWFHGKLSRREAEALLQLNGDFLVRESTTTPGQYVLTGLQSGQPKHLLLVDPEGVVRTKDHRFESVSHLISYHMDNHLPIISAGSELCLQQPVERKL

SEQ ID NO: 7 - DNA, 1462 bp, Mus musculus
cggaaccagatcggcccgcggtgcggtgcggagactccatgagaccctggacatgaacaagctgagtggaggcggcgggcgcaggactcgggtagaagggggccagctggggggcgaggagtggaccagacacgggagctttgtcaataagcccacacgaggctggctgcatcccaacgacaaagtcatgggacctggggtttcctacttggttcggtacatgggctgtgtggaggtcttacagtcaatgcgagcccttgacttcaatacccggactcaggtcaccagggaggccatcagtttggtgtgtgaagctgtgcctggtgccaaaggggcgacaaggaggagaaagccttgtagccgcccactcagctccatcctggggaggagtaacctgaagtttgctggaatgccaatcactctcactgtgtctaccagcagccttaacctcatggcagccgactgcaaacagatcattgccaaccatcacatgcaatctatctctttcgcgtccggtggggatccggacacagctgagtatgttgcctatgttgccaaagaccctgtgaatcagagagcctgccatatcctggagtgtcctgaagggcttgctcaggatgtcatcagcaccatcgggcaggcctttgagttgcgcttcaaacagtatctcaggaatccaccgaagctggtcaccccccatgacaggatggctggctttgatggctcagcttgggatgaggaggaagaagagccccctgaccatcagtactacaatgactttccagggaaggaaccccctcttggtggggtggtagatatgaggcttcgggaaggggctgctcgacccactctgcctagtgcccagatgtccagccacttgggagctacactgcctatagggcagcatgctgcaggagaccatgaagtccgtaaacagatgttgcctccgccgccttgcccaggcagagaactcttcgatgacccctcctatgtcaacatccagaatctagacaaggcccggcaggctgggggtggggctgggcccccaaatccttctcttaatggcagtgcaccccgagacctttttgacatgaagccctttgaagatgcacttcgggtgccacccccaccgcagtccatgtccatggctgagcagctgcaaggggagccctggttccacgggaagctgagccggagggaggccgaggcgctgctgcagctcaatggtgacttcttggtgcgagagagcacgaccacgcctggccagtatgtgctcactggcctgcagagtgggcagcccaagcacttgctgctggtggaccctgaaggtgtggttcggacaaaggatcaccgctttgagagtgtcagtcacctgatcagctaccacatggacaatcacttgcccatcatctctgcgggcagcgaactgtgcctacagcaacccgtggatcggaaagtgtga

SEQ ID NO: 8 - protein, 469 aa, Mus musculus
MNKLSGGGGRRTRVEGGQLGGEEWTRHGSFVNKPTRGWLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRNPPKLVTPHDRMAGFDGSAWDEEEEEPPDHQYYNDFPGKEPPLGGVVDMRLREGAARPTLPSAQMSSHLGATLPIGQHAAGDHEVRKQMLPPPPCPGRELFDDPSYVNIQNLDKARQAGGGAGPPNPSLNGSAPRDLFDMKPFEDALRVPPPPQSMSMAEQLQGEPWFHGKLSRREAEALLQLNGDFLVRESTTTPGQYVLTGLQSGQPKHLLLVDPEGVVRTKDHRFESVSHLISYHMDNHLPIISAGSELCLQQPVDRKV

SEQ ID NO: 9 - DNA, 1739 bp, Mus musculus
atggatcttctaccccccaagccgaagtacaacccacttcggaatgagtctctgtcatcgctggaggagggggcttcggggtctacccctccggaggagctaccttccccatcagcttcatccctgggacccattctgcctcctctgccgggggacgatagtccgactaccctgtgttccttctttccccggatgagcaacctgaagctggccaatcctgctggggggcgcctggggcctaaaggggagccaggaaaggctgctgaagatggggaagggagtgcaggggcagcccttcgggactcaggcctcttgcccctcctccaggacatgaacaagctgagtggaggcggcgggcgcaggactcgggtagaagggggccagctggggggcgaggagtggaccagacacgggagctttgtcaataagcccacacgaggctggctgcatcccaacgacaaagtcatgggacctggggtttcctacttggttcggtacatgggctgtgtggaggtcttacagtcaatgcgagcccttgacttcaatacccggactcaggtcaccagggaggccatcagtttggtgtgtgaagctgtgcctggtgccaaaggggcgacaaggaggagaaagccttgtagccgcccactcagctccatcctggggaggagtaacctgaagtttgctggaatgccaatcactctcactgtgtctaccagcagccttaacctcatggcagccgactgcaaacagatcattgccaaccatcacatgcaatctatctctttcgcgtccggtggggatccggacacagctgagtatgttgcctatgttgccaaagaccctgtgaatcagagagcctgccatatcctggagtgtcctgaagggcttgctcaggatgtcatcagcaccatcgggcaggcctttgagttgcgcttcaaacagtatctcaggaatccaccgaagctggtcaccccccatgacaggatggctggctttgatggctcagcttgggatgaggaggaagaagagccccctgaccatcagtactacaatgactttccagggaaggaaccccctcttggtggggtggtagatatgaggcttcgggaaggggctgctcgacccactctgcctagtgcccagatgtccagccacttgggagctacactgcctatagggcagcatgctgcaggagaccatgaagtccgtaaacagatgttgcctccgccgccttgcccaggcagagaactcttcgatgacccctcctatgtcaacatccagaatctagacaaggcccggcaggctgggggtggggctgggcccccaaatccttctcttaatggcagtgcaccccgagacctttttgacatgaagccctttgaagatgcacttcgggtgccacccccaccgcagtccatgtccatggctgagcagctgcaaggggagccctggttccacgggaagctgagccggagggaggccgaggcgctgctgcagctcaatggtgacttcttggtgcgagagagcacgaccacgcctggccagtatgtgctcactggcctgcagagtgggcagcccaagcacttgctgctggtggaccctgaaggtgtggttcggacaaaggatcaccgctttgagagtgtcagtcacctgatcagctaccacatggacaatcacttgcccatcatctctgcgggcagcgaactgtgcctacagcaacccgtggatcggaaagtgga

SEQ ID NO: 10 - protein, 579 aa, Mus musculus
MDLLPPKPKYNPLRNESLSSLEEGASGSTPPEELPSPSASSLGPILPPLPGDDSPTTLCSFFPRMSNLKLANPAGGRLGPKGEPGKAAEDGEGSAGAALRDSGLLPLLQDMNKLSGGGGRRTRVEGGQLGGEEWTRHGSFVNKPTRGWLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRNPPKLVTPHDRMAGFDGSAWDEEEEEPPDHQYYNDFPGKEPPLGGVVDMRLREGAARPTLPSAQMSSHLGATLPIGQHAAGDHEVRKQMLPPPPCPGRELFDDPSYVNIQNLDKARQAGGGAGPPNPSLNGSAPRDLFDMKPFEDALRVPPPPQSMSMAEQLQGEPWFHGKLSRREAEALLQLNGDFLVRESTTTPGQYVLTGLQSGQPKHLLLVDPEGVVRTKDHRFESVSHLISYHMDNHLPIISAGSELCLQQPVDRKV

SEQ ID NO: 11 - protein, 11 aa, Artificial Sequence
  RTK binding site variant
LPVPEYINQSV

SEQ ID NO: 12 - protein, 11 aa, Artificial Sequence
  RTK binding site variant
LPVPEFINQSV

SEQ ID NO: 13 - protein, 11 aa, Artificial Sequence
  RTK binding site variant
IENPQYFSDAC

SEQ ID NO: 14 - protein, 11 aa, Artificial Sequence
  RTK binding site variant
IENPQFFSDAC

SEQ ID NO: 15 - protein, 11 aa, Artificial Sequence
  RTK binding site variant
AENAEYLRVAP

SEQ ID NO: 16 - protein, 9 aa, Artificial Sequence
  RTK variants
NATFVNVKC